(19) United States
(12) Patent Application Publication
(10) Pub. No.: US 2020/0375872 A1
(43) Pub. Date: Dec. 3, 2020

(54) HAIR CARE SYSTEMS AND METHODS FOR DELIVERY OF CUSTOMIZED HAIR CARE PRODUCTS

(71) Applicant: L'OREAL, Paris (FR)

(72) Inventors: Eugene HARDY, Old Bridge, NJ (US); Ma Angellica Mangaban Ceballos, Hackensack, NJ (US)

(21) Appl. No.: 16/888,665

(22) Filed: May 30, 2020

Related U.S. Application Data
(60) Provisional application No. 62/855,818, filed on May 31, 2019.

(51) Int. Cl. A61K 8/73; A61K 8/96; A61K 8/19; A61K 8/34; A61K 8/9794; A61K 8/92; A61Q 5/02; A61Q 5/12; A61K 47/22

(52) U.S. Cl. CPC A61K 8/732; A61K 8/965; A61K 8/19; A61K 8/342; A61K 8/9794; A61K 2800/30; A61Q 5/02; A61Q 5/12; A61K 47/22; A61K 2800/5426; A61K 8/922

(57) ABSTRACT

A system for formulating a hair care product includes a hair product formulary schedule that includes hair care formulation recipes for combining aqueous base compositions and water dispersible cosmeticity booster compositions that include active ingredients corresponding to hair target attributes, which are in some embodiments high naturality active ingredients. The prepared compositions may be used alone, or in various combinations, to provide hair care regimens for addressing and achieving one or more hair care or styling attributes.

### PRIORITY

This application claims the benefit under 35 U.S.C. § 119(e) and priority to U.S. Provisional Patent Application Ser. No. 62/855,818, filed on May 31, 2019, entitled HAIR CARE SYSTEMS AND METHODS FOR DELIVERY OF CUSTOMIZED HAIR CARE PRODUCTS, the entirety of which is incorporated by reference herein.

### FIELD OF TECHNOLOGY

The instant disclosure relates to systems and methods for formulating hair care compositions. The disclosure also provides hair care compositions. The compositions include materials formulated into hair cleansing, conditioning or cuticle sealing bases and incorporating hair benefit booster formulas, which include, in some embodiments, materials that are minimally processed with high naturality. The compositions are prepared by methods that enable formulation of hair care products and combinations of products just prior to treatment application and based on selection of specific target hair target attributes, allowing for creation of formulations that deliver customized sensory experiences and hair benefits to a consumer.

### BACKGROUND

Personalized and customized hair care systems are desirable for consumers to provide one or an array of possible hair treatments that meets the hair care needs and styling goals of the individual consumer.

There are several examples of specialized, customized and personalized treatments for hair. Some services allow consumers to configure a regimen using off the shelf products based on consumer preferences, including Shtrands™ and Form Beauty™. An example of a specialized product line includes the Matrix Biolage R.A.W.™ product line which offers a suite of shampoo, conditioner, mask and styling products and regimens for hair care that are natural-ingredient focused, where one or more products or regimes may be selected to address consumers' specifically identified needs and are purchased for home use. Examples of customized product lines driven by consumer data inputs include Prose™, Function of Beauty™, Belle Bar™ Organic Customizable Hair Masks, Schwarzkopf's BC Fibre Clinix™, and Ouai™ Hair Consult, which enable consumers to obtain one or a suite of hair care products that are specifically formulated for the consumer based on proprietary algorithms that correlate consumer hair condition and style objective inputs with arrays of available ingredients to formulate custom products that are delivered to the consumer for home use. And Schwarzkopf's™ SalonLab™ analyzer offers a hair scanning technology that analyzes hair condition and color, and then combined with customer inputs and preferences, provides personalized custom treatments for home use.

Each of these approaches provide enhanced opportunities for consumers to obtain hair products with components that are selected with amounts and types of ingredients dialed in to meet their needs. However, what remains unaddressed is the increasing consumer focus on real-time fresh hair treatment products that are natural and sustainable and retain their active and “live” effectiveness that cannot be obtained in a shelf stored product whether dispensed in a salon or mailed to a consumer. Thus, there remains a need to provide custom hair care product solutions employing salon quality and strength, high-end and natural active ingredients that offer improvement of a consumer's hair condition and achievement of the consumer's styling goals.

### BRIEF SUMMARY

According to various embodiments, a system and method for formulating hair care products is provided.

According to various embodiments, a system and method for formulating hair care products is provided.

In an exemplary embodiment, a system for formulating a hair care product includes a hair product formulary schedule that includes a plurality of hair care formulation recipes. The recipes include directions for combining one of a plurality of aqueous base compositions with one or more of a plurality of water dispersible cosmeticity booster compositions. In some particular embodiments, one or more of the base and booster compositions include high naturality active ingredients, wherein one or more of the booster compositions may include select high naturality active ingredients corresponding to specific hair target attributes. The prepared compositions may be used alone, or in various combinations, to provide hair care regimens for addressing and achieving one or more hair care or styling attributes. The plurality of aqueous base compositions includes at least one cleansing base and at least one conditioning base, each aqueous base composition disposed in a separate dispensing container. Each of the plurality of water dispersible cosmeticity booster compositions is readily dispersible into the base compositions and each is disposed in a separate dispensing container.

In some embodiments, the system includes one or more additional base compositions.

In some particular embodiments, the plurality of aqueous base compositions includes a cleansing base composition that comprises at least one alkyl glucoside present from about 1% to about 15%, and in some particular embodiments present from about 9% to about 10%, and at least one cationic polymer present from about 0.01% to about 3%, and in some particular embodiments present from about 0.05% to about 0.15%. In some such embodiments, the composition includes an additive comprising an active ingredient that comprises an organic acid, for example a beta hydroxy acid present from about 0.1% to about 0.3%, and in some particular embodiments present from about 0.25% to about 0.75%, wherein all percentages are by weight based upon the weight of the booster composition.

In an exemplary embodiment, a cleansing base composition according to the disclosure comprises coco-glucoside hydroxypropyl, guar hydroxypropyltrimonium chloride, an organic acid, for example, salicylic acid, and water.

In some particular embodiments, the plurality of aqueous base compositions includes a conditioning base composition that comprises at least one cationic surfactant including cationizable surfactants together with an acid neutralizer, present from about 0.5 to about 5%, and in some particular embodiments present from about 2.25% to about 2.75%, and when present an acid neutralizer present from about 0.25 to about 0.5%, at least one fatty alcohol, present from about 0.5% to about 15%, and in some particular embodiments present from about 2% to about 4%. In some such embodiments, the composition includes an additive comprising an active ingredient that comprises an organic acid, for example a beta hydroxy acid, present from about 0.1% to about 0.3%, and in some particular embodiments present from about 0.25% to about 0.75%, wherein all percentages are by weight based upon the weight of the booster composition.

In an exemplary embodiment, a conditioning base composition according to the disclosure comprises at least one cationic surfactant comprising a cationizable surfactant comprising BRASSICAMIDOPROPYL DIMETHYLAMINE together with an acid neutralizer comprising tartaric acid, at least one fatty alcohol comprising cetearyl alcohol, and at an active comprising an organic acid, for example, salicylic acid, and water.

In some embodiments, the plurality of water dispersible cosmeticity booster compositions correspond, respectively, to hair target attributes that include exfoliating, volumizing, plumping, smoothing, softening, repairing/softening, and scenting.

In some embodiments, the plurality of water dispersible cosmeticity booster compositions includes two or more of:
(i) the booster composition corresponding to exfoliating that includes a powder, present from about 1% to about 15%, and in some particular embodiments present from about 5% to about 7%, at least one clay, present from about 10% to about 40%, and in some particular embodiments present from about 20% to about 30%, at least one filler, present from about 45% to about 90%, and in some particular embodiments present from about 65% to about 70%; and
(ii) the booster composition corresponding to volumizing that includes sodium bicarbonate, present from about 10% to about 30%, and in some particular embodiments present from about 18% to about 22%, an organic acid, present from about 2% to about 15%, and in some particular embodiments present from about 6% to about 8%, and starch, present from about 40% to about 90%, and in some particular embodiments present from about 70% to about 75%; and
(iii) the booster composition corresponding to plumping that includes at least one polysaccharide gellifying thickener, present from about 0.5% to about 5%, and in some particular embodiments present from about 0.5% to about 1%, aloe, present from about 40% to about 75%, and in some particular embodiments present from about 62% to about 68%, one or a combination of polyols, each present from about 0.5% to about 20%, and in some particular embodiments each present from about 1.5% to about 10%, and optionally an active comprising an organic acid, for example a beta hydroxy acid, present from about 0.1% to about 0.3%, and in some particular embodiments present from about 0.1% to about 0.3%; and
(iv) the booster composition corresponding to smoothing that includes at least one gum, present from about 0.5% to about 5%, and at least one hydroxy group containing compound selected from polyols and longer chain monoalcohols (C8 to C30), present from 0.1% to about 20%; and
(v) the booster composition corresponding to repairing butter that includes an anhydrous blend of plant/vegetable oils, comprising sunflower seed oil, coconut oil, avocado oil, each present from about 0.001% to about 90%, sunflower seed oil present from about 0.001% to about 1%, and in some particular embodiments present from about 0.007% to about 0.009%, coconut oil present from about 40% to about 90%, and in some particular embodiments present from about 75% to about 80%, avocado oil present from about 5% to about 35%, and in some particular embodiments present from about 18% to about 22%, and optionally at least one active comprising a vitamin, present from about 0.00001% to about 1%, and in some particular embodiments present from about 0.00003% to about 0.00007%; and
(vi) the booster composition corresponding to repairing/softening oil that includes an anhydrous blend comprising at least one plant oil, present from about 0.01% to about 3%, and in some particular embodiments present from about 1.0% to about 1.2%, the Caryocar Brasiliense oil present from about 0.01% to about 1.5%, and in some particular embodiments present from about 0.8% to about 1.2%, the jojoba oil present from about 0.1% to about 3%, and in some particular embodiments present from about 0.8% to about 1.2%, at least one polar emollient ester, present from about 50% to about 99%, and in some particular embodiments present from about 95% to about 99%, and optionally an active, present from about 0.0001% to about 1%, and in some particular embodiments present from about 0.00003% to about 0.00007%; and
(vii) the booster composition corresponding to scenting that includes an essentially anhydrous or anhydrous blend of at least one fragrance with at least one polar emollient, comprising isopropyl palmitate, present from about 50% to about 99%, and in some particular embodiments present from about 92% to about 98%, the balance comprising at least one fragrance; and wherein all percentages are by weight based upon the weight of the booster composition.

In some particular embodiments, the plurality of water dispersible cosmeticity booster compositions includes two or more of:
(i) the booster composition corresponding to exfoliating that comprises at least one or a combination of cosmetic powders and clays; and
(ii) the booster composition corresponding to volumizing that comprises at least one starch; and
(iii) the booster composition corresponding to plumping that comprises aloe, an organic acid, for example, salicylic acid, and at least one humectant, and water; and
(iv) the booster composition corresponding to smoothing that comprises at least one plant gum, at least one fatty alcohol emollient and at least one humectant, and water; and
(v) the booster composition corresponding to softening that comprises at least one polar emollient ester and at least one fruit oil; and
(vi) the booster composition corresponding to repairing/softening that comprises a combination of plant or fruit oil comprising one or more of avocado oil and coconut oil; and
(vii) the booster composition corresponding to scenting that includes an essentially anhydrous or anhydrous blend of a fragrance with at least one polar emollient.

According to some embodiments, the system for formulating a hair care product includes each of the aqueous base compositions and each of the water dispersible cosmeticity booster compositions disposed in a dispensing container that is adapted to dispense a fixed unit of the composition. According to such embodiments, each hair care formulation recipe in the hair product formulary schedule includes instructions to dispense a specific number of pre-determined fixed units of each composition into a mixing vessel. According to such embodiments, each fixed unit of composition corresponds with a predetermined amount/concentration by weight of actives, with reference to the conversions as set forth in Example 29.

According to some embodiments, the system for formulating a hair care product includes in the hair product formulary schedule hair cleansing formulation recipes selected from the group consisting of:
(i) a hair cleansing formulation comprising a predetermined number of dispensed units of a paraben-free cleansing base, a predetermined number of dispensed units of a volumizing booster, and a predetermined number of dispensed units of a plumping booster;
(ii) a hair cleansing formulation comprising a predetermined number of dispensed units of a paraben-free cleansing base, and a predetermined number of dispensed units of a repairing/softening booster;
(iii) a hair cleansing formulation comprising a predetermined number of dispensed units of a paraben-free cleansing base, and a predetermined number of dispensed units of a smoothing booster;
(iv) a hair cleansing formulation comprising a predetermined number of dispensed units of a paraben-free cleansing base, and a predetermined number of dispensed units of n exfoliating booster;
(v) a hair cleansing formulation comprising a predetermined number of dispensed units of a paraben-free cleansing base, a volumizing booster, and a predetermined number of dispensed units of a plumping booster;
(vi) a hair cleansing formulation comprising a predetermined number of dispensed units of a paraben-free cleansing base, a predetermined number of dispensed units of a volumizing booster, and a predetermined number of dispensed units of a plumping booster;
(vii) a hair cleansing formulation comprising a predetermined number of dispensed units of a paraben-free cleansing base, a predetermined number of dispensed units of a smoothing booster and a predetermined number of dispensed units of a softening booster;
(viii) a hair cleansing formulation comprising a predetermined number of dispensed units of a paraben-free cleansing base, a predetermined number of dispensed units of a smoothing booster, a predetermined number of dispensed units of a softening booster, and a predetermined number of dispensed units of a volumizing booster; and
(ix) a hair cleansing formulation comprising a predetermined number of dispensed units of a paraben-free cleansing base, a predetermined number of dispensed units of a smoothing booster, and a predetermined number of dispensed units of a softening booster.

According to some embodiments, the system for formulating a hair care product includes in the hair product formulary schedule hair cleansing formulation recipes selected from the group consisting of:
(i) a hair conditioning formulation comprising a predetermined number of dispensed units of a paraben-free conditioning base, a predetermined number of dispensed units of a repairing/softening booster, and a predetermined number of dispensed units of a plumping booster;
(ii) a hair conditioning formulation comprising a predetermined number of dispensed units of a paraben-free conditioning base and a predetermined number of dispensed units of a smoothing booster;
(iii) a hair conditioning formulation comprising a predetermined number of dispensed units of a paraben-free conditioning base and a predetermined number of dispensed units of a volumizing booster;
(iv) a hair conditioning formulation comprising a predetermined number of dispensed units of a paraben-free conditioning base, a predetermined number of dispensed units of a smoothing booster, a plumping booster, and a predetermined number of dispensed units of a repairing/softening booster;
(v) a hair conditioning formulation comprising a predetermined number of dispensed units of a paraben-free conditioning base and a repairing/softening booster;
(vi) a hair conditioning formulation comprising a predetermined number of dispensed units of a paraben-free conditioning base and a smoothing booster.

In another exemplary embodiment, a method for delivering hair care includes the steps of:
(I) Providing in any order:
   a) a hair product formulary schedule comprising a plurality of hair care formulation recipes, each hair care formulation recipe including instructions for combining one of a plurality of aqueous base compositions, and one or a combination of water dispersible cosmeticity booster compositions;
   b) an aqueous base composition disposed in a dispensing container, the dispensing container adapted to dispense a fixed unit of aqueous base composition, the aqueous base a cleansing base composition that comprises one of:
      i) a cleansing base composition that comprises at least one alkyl glucoside at least one cationic polymer, water and optionally at least one active; and
      ii) a conditioning base composition that comprises at least one cationic surfactant including cationizable surfactants together with an acid neutralizer, at least one fatty alcohol, and water, and optionally at least one active;
      wherein all percentages are by weight based upon the weight of the aqueous base composition;
   c) a plurality of water dispersible cosmeticity booster compositions, each being readily dispersible into the base compositions and each disposed in a separate dispensing container, the plurality of water dispersible cosmeticity booster compositions comprising active ingredients corresponding to hair target attributes that include exfoliating, volumizing, plumping, smoothing, softening, repairing/softening, and scenting, the plurality of water dispersible cosmeticity booster compositions comprising two or more of:
      (i) the booster composition corresponding to exfoliating that includes a powder, at least one clay, at least one filler, and starch; and
      (ii) the booster composition corresponding to volumizing that includes sodium bicarbonate, an organic acid, and starch; and
      (iii) the booster composition corresponding to plumping that includes at least one polymeric thickener, in some embodiments comprising a polysaccharide, in some embodiments comprising a polysaccharide gellifying thickener, together with aloe, one or a combination of polyols, and optionally an active comprising an organic acid, for example a beta hydroxy acid, and water; and
      (iv) the booster composition corresponding to smoothing that includes at least one gum, and at least one hydroxy group containing compound selected from polyols, longer chain monoalcohols (C8 to C30) which include fatty alcohols, and combinations thereof, and water; and
      (v) the booster composition corresponding to repairing butter that includes an anhydrous blend of plant/vegetable oils, and optionally at least one active comprising a vitamin; and
      (vi) the booster composition corresponding to repairing/softening oil that includes an anhydrous blend comprising at least one plant oil, at least one polar emollient ester, and optionally at least one active comprising a vitamin, comprising tocopherol; and
      (vii) the booster composition corresponding to scenting that includes an essentially anhydrous or anhydrous blend of a fragrance with polar emollient selected from isopropyl palmitate, isopropyl myristate, and combinations thereof
(I) Preparing a hair care formulation, including:
   d) Selecting a first hair care formulation recipe from the hair product formulary schedule;
   e) Dispensing into a mixing vessel a number of units of the aqueous base composition as set forth in the recipe;
   f) Dispensing into the mixing vessel a number of units of each of one or a combination of booster compositions as set forth in the recipe, alternately mixing after addition of each booster composition; and
   g) Completing any additional processing step as set forth in the recipe;
(II) Delivery of hair care, including:
   h) Applying the hair care formulation to one of the scalp, the hair ends, the hair roots, and combinations thereof.

In some embodiments, the method for delivering hair care includes repeating the steps at least two times to form at least a pair of hair care formulations selected from at least one cleansing and at least one conditioning product, or at least two cleansing products, or at least two conditioning products.

The methods enable formulation of hair care products and combinations of products just prior to treatment application and based on selection of specific target hair target attributes, allowing for creation of formulations that deliver customized sensory experiences and hair benefits to a consumer.

Other features and advantages of the present invention will be apparent from the following more detailed description of the preferred embodiment which illustrates, by way of example, the principles of the invention.

### BRIEF DESCRIPTION OF THE DRAWINGS

This application contains at least one drawing executed in color. Copies of this patent or patent application publication with color drawing(s) will be provided by the Office upon request and payment of the necessary fee.

Features and advantages of the general inventive concepts will become apparent from the following detailed description made with reference to the accompanying drawings.

FIG. 1 includes two photographic images of hair treated with conventional compositions (on the left) and hair of the same subject treated with one or more inventive compositions according to the disclosure (on the right);

FIG. 2 includes two photographic images of hair treated with conventional compositions (on the left) and hair of the same subject treated with one or more inventive compositions according to the disclosure (on the right);

FIG. 3 includes two photographic images of hair treated with conventional compositions (on the left) and hair of the same subject treated with one or more inventive compositions according to the disclosure (on the right);

FIG. 4 includes two photographic images of hair treated with conventional compositions (on the left) and hair of the same subject treated with one or more inventive compositions according to the disclosure (on the right);

FIG. 5 includes two photographic images of hair treated with conventional compositions (on the left) and hair of the same subject treated with one or more inventive compositions according to the disclosure (on the right);

FIG. 6 includes two photographic images of hair treated with conventional compositions (on the left) and hair of the same subject treated with one or more inventive compositions according to the disclosure (on the right);

FIG. 7 includes a photographic image of hair treated one or more inventive compositions according to the disclosure;

FIG. 8 includes two photographic images of hair treated with conventional compositions (on the left) and hair of the same subject treated with one or more inventive compositions according to the disclosure (on the right);

FIG. 9 includes two photographic images of hair treated with conventional compositions (on the left) and hair of the same subject treated with one or more inventive compositions according to the disclosure (on the right);

FIG. 10 includes a series photographic images of several subjects, each having hair treated with conventional compositions (on the top) and hair of the same subject treated with one or more inventive compositions according to the disclosure (on the bottom);

FIG. 11 includes a series photographic images of several subjects, each having hair treated with conventional compositions (on the top) and hair of the same subject treated with one or more inventive compositions according to the disclosure (on the bottom); and

FIG. 12 includes a series photographic images of several subjects, each having hair treated with conventional compositions (on the top) and hair of the same subject treated with one or more inventive compositions according to the disclosure (on the bottom).

### DETAILED DESCRIPTION

The use of a variety of high naturality raw materials utilized in cosmetic formulas at efficacious levels is not new. Indeed, such materials are of increasing interest to consumers who seek ever more natural and sustainable products. However, compositions that employ such materials are challenged with complexities of delivering similar cosmeticity as compared with traditional cleansing and conditioning products. Typically, high naturality ingredients can prove to have poor stability/compatibility behavior, and experience changes in textural or sensorial attributes throughout extended product usage as well long-term deterioration of micro stability. In order to minimize or improve those shortcomings, additional materials such as stabilizers, structurants, rheology modifiers and processing aids are incorporated. Use of these components may help to overcome the detrimental deterioration of some ingredients, but they increase the cost and complexity of the formulas, can result in reduced hair cosmeticity or other sensory attribute limitations, and in some instances diminish or destroy the benefit sought to be conferred by the ingredient.

Hair care recipes that could be created just prior to point of use (single application) allows for the combination of high levels of cosmetic raw materials with high naturality that can provide both high levels of hair cosmeticity and transformative textures (self-foaming, generation of paste or cream like appearance, etc.). Point of use compositions could provide consumers with hair care solutions that are handcrafted and personal to provide a customized experience with transformative textures and sensory experiences (including addition of hair treatment and fragrance boosters) during mixing, application and post-application, taking maximal advantage of the key high naturality ingredients.

The system and method, according to the present disclosure, includes a method wherein a custom configured hair care can be delivered by selection of one or a combination of hair products, for example hair cleansing and hair conditioning, formulations that address hair target attributes. The system according to the instant disclosure includes, in some embodiments, materials that are minimally processed with high naturality. The system provides a hair product formulary schedule of hair product formulation recipes, each tailored to address one or a combination of hair target attributes. They system further includes suites of compositions that include aqueous base compositions, for example aqueous bases for cleansers and for conditioners, and water dispersible cosmeticity booster compositions, for example boosters that target hair volume, or smoothness. In accordance with certain embodiments, some of which are exemplified herein, the system enables in-salon formulation of hair care products that take advantage of the high naturality ingredients in the compositions without the negatives associated with longer shelf preservation requirements for prepackaged products. And the system enables production of custom selected regimens that include combinations of the recipe formulations as well as the base and booster compositions.

As used herein, the term “high naturality materials” means cosmetically acceptable materials that are one or more of directly obtained from nature, are obtained from nature with minimal processing, and are derivatives of materials that are obtained from nature.

As used herein, the term “hair product formulary schedule” means a listing of formulations of hair care compositions that comprise single or combinations of compositions according to the disclosure, wherein the formulations are directed to hair care that includes but is not limited to cleansing, conditioning, masking, and sealing.

As used herein, the term “hair care formulation recipes” means formulations of hair care compositions that comprise single or combinations of compositions according to the disclosure, wherein the formulations are directed to hair care that includes but is not limited to cleansing, conditioning, masking, and sealing

As used herein, the term “aqueous base compositions” means formulations of hair care compositions that comprise single or combinations of compositions according to the disclosure, wherein the formulations are directed to hair cleansing.

As used herein, the term “anhydrous” refers to compositions or blends thereof which are essentially free from or devoid of water.

As used herein, the term “water dispersible cosmeticity booster” compositions means compositions that can be dispersed or dissolved in an aqueous system.

As used herein, the term “cosmeticity” means the effect of a cosmetic composition to confer an improvement to or correction of a skin property such as smoothness, texture, volume, softness, dryness, presence of fine lines and wrinkles, etc.

As used herein, the term “hair target attribute” means a property or feature of the keratinous substrate, particularly hair, that is sought to be conferred, altered or improved. In one example, a target attribute is exfoliation, for example exfoliate of the keratinous tissue of the scalp at the root of hair. As used herein, the term exfoliating means

As used herein, the term volumizing means. As used herein, the term plumping means. As used herein, the term smoothing means. As used herein, the term softening means. As used herein, the term repairing/softening means. As used herein, the term scenting means

As used herein, the term “adapted to dispense a fixed unit of cleansing base composition” means fitted with or provided with instructions for delivering a controlled unit measure of an ingredient, for example using a conventional measuring device selected from a measuring cup, scoop or spoon, a graduated vial, tube, of dropper, or employing a pump mechanism that delivers a fixed volume aliquot of material.

According to various embodiments, a system and method for formulating hair care products is provided.

In an exemplary embodiment, a system for formulating a hair care product includes a hair product formulary schedule that includes a plurality of hair care formulation recipes. The recipes include directions for combining one of a plurality of aqueous base compositions with one or more of a plurality of water dispersible cosmeticity booster compositions. The base and booster compositions include in some embodiments high naturality active ingredients, and the booster compositions include in some embodiments select high naturality active ingredients corresponding to specific hair target attributes. The prepared compositions may be used alone, or in various combinations, to provide hair care regimens for addressing and achieving one or more hair care or styling attributes. The plurality of aqueous base compositions includes at least one cleansing base and at least one conditioning base, each aqueous base composition disposed in a separate dispensing container. Each of the plurality of water dispersible cosmeticity booster compositions is readily dispersible into the base compositions and each is disposed in a separate dispensing container.

It will be understood that in some embodiments, the reagents/components of compositions and formulations as described herein may not include any or may only include some high naturality materials, while in other embodiments, including some that are exemplified herein, the reagents/components of compositions and formulations hereof may be selected from or comprise high naturality materials, and though in some exemplified embodiments, the disclosure references ingredients that are directly obtained from nature, in other embodiments such ingredients may be obtained from sources that are obtained from nature with minimal processing, or are derivatives of materials that are obtained from nature

Base and Booster Compositions

Aqueous Base Compositions

In some embodiments, the plurality of aqueous base compositions includes one of each of a cleansing base composition and a conditioning base composition. In some embodiments, the system includes one or more additional base compositions, in some embodiments wherein the additional base compositions include vinegar rinse.

Cleansing Base Compositions

In the various embodiments, the plurality of aqueous base compositions includes at least one cleansing base composition.

In some particular embodiments, the plurality of aqueous base compositions includes a cleansing base composition that comprises at least one alkyl glucoside, and at least one cationic polymer. In some such embodiments, the composition includes an additive comprising an active ingredient that comprises an organic acid, for example a beta hydroxy acid.

In some particular embodiments, the plurality of aqueous base compositions includes a cleansing base composition that comprises at least one alkyl glucoside present from about 1% to about 15%, and in some particular embodiments present from about 9% to about 10%, and at least one cationic polymer present from about 0.01% to about 3%, and in some particular embodiments present from about 0.05% to about 0.15%. In some such embodiments, the composition includes an additive comprising an active ingredient that comprises an organic acid, for example a beta hydroxy acid present from about 0.1% to about 0.3%, and in some particular embodiments present from about 0.25% to about 0.75%, wherein all percentages are by weight based upon the weight of the booster composition. In an exemplary embodiment, a cleansing base composition according to the disclosure comprises coco-glucoside hydroxypropyl, guar hydroxypropyltrimonium chloride, an organic acid, for example, salicylic acid, and water.

Active ingredients corresponding to cleansing compositions include, in some embodiments include: at least one alkyl glucoside selected from Arachidyl Glucoside, C12-20 Alkyl Glucoside, Caprylyl/Capryl Glucoside, Cetearyl Glucoside, Coco-Glucoside, Ethyl Glucoside and Lauryl Glucoside, Decyl Glucoside and combinations thereof; and at least one cationic polymer selected from Polyquaternium-6, polyquaternium-10, polyquaternium-4, and hydroxypropyl guar hydroxypropyltrimonium chloride, and combinations thereof. In some embodiments, the compositions include an additive that includes at least one organic acid, for example a beta-hydroxy acid selected from salicylic acid, propionic acid, beta-hydroybutyric acid, beta-hydroxy beata-methylbutyric acid, carnitine, and combinations thereof.

Conditioning Base Compositions

In the various embodiments, the plurality of aqueous base compositions includes at least one conditioning base composition.

In some particular embodiments, the plurality of aqueous base compositions includes a conditioning base composition that comprises at least one cationic surfactant including cationizable surfactants together with an acid neutralizer, and at least one fatty alcohol. In some such embodiments, the composition includes an additive comprising an active ingredient that comprises a beta hydroxy acid.

In some particular embodiments, the plurality of aqueous base compositions includes a conditioning base composition that comprises at least one cationic surfactant including cationizable surfactants together with an acid neutralizer, present from about 0.5 to about 5%, and in some particular embodiments present from about 2.25% to about 2.75%, and when present an acid neutralizer present from about 0.25 to about 0.5%, at least one fatty alcohol, present from about 0.5% to about 15%, and in some particular embodiments present from about 2% to about 4%. In some such embodiments, the composition includes an additive comprising an active ingredient that comprises a beta hydroxy acid, present from about 0.1% to about 0.3%, and in some particular embodiments present from about 0.25% to about 0.75%, wherein all percentages are by weight based upon the weight of the booster composition.

In an exemplary embodiment, a conditioning base composition according to the disclosure comprises at least one cationic surfactant comprising a cationizable surfactant comprising BRASSICAMIDOPROPYL DIMETHYLAMINE together with an acid neutralizer comprising tartaric acid, at least one fatty alcohol comprising cetearyl alcohol, and at an active comprising an organic acid, for example, salicylic acid.

Cosmeticity Booster Compositions

In some embodiments, the plurality of water dispersible cosmeticity booster compositions correspond, respectively, to hair target attributes that include exfoliating, volumizing, plumping, smoothing, softening, repairing/softening, and scenting.

In some embodiments, the plurality of water dispersible cosmeticity booster compositions includes two or more of:

(i) the booster composition corresponding to exfoliating; and

(ii) the booster composition corresponding to volumizing; and

(iii) the booster composition corresponding to plumping; and

(iv) the booster composition corresponding to smoothing; and

(v) the booster composition corresponding to repairing/softening; and

(vi) the booster composition corresponding to scenting.

Exfoliating Hair Target Attribute Booster

In the various embodiments, the plurality of water dispersible cosmeticity booster compositions includes at least one booster composition corresponding to exfoliating.

In some particular embodiments, the plurality of water dispersible cosmeticity booster compositions includes the booster composition corresponding to exfoliating that includes a powder, at least one clay, at least one filler, and starch.

In some particular embodiments, the plurality of water dispersible cosmeticity booster compositions includes the booster composition corresponding to exfoliating that includes a powder, present from about 1% to about 15%, and in some particular embodiments present from about 5% to about 7%, at least one clay, present from about 10% to about 40%, and in some particular embodiments present from about 20% to about 30%, at least one filler, present from about 45% to about 90%, and in some particular embodiments present from about 65% to about 70%.

In an exemplary embodiment, the booster composition corresponding to exfoliating that includes a powder comprising argania spinosa shell powder, at least one clay, comprising kaolin, at least one filler, comprising talc.

Volumizing Hair Target Attribute Booster

In the various embodiments, the plurality of water dispersible cosmeticity booster compositions includes at least one booster composition corresponding to volumizing.

In some particular embodiments, the plurality of water dispersible cosmeticity booster compositions includes the booster composition corresponding to volumizing that includes sodium bicarbonate, an organic acid, and starch.

In some particular embodiments, the plurality of water dispersible cosmeticity booster compositions includes the booster composition corresponding to volumizing that includes sodium bicarbonate, present from about 10% to about 30%, and in some particular embodiments present from about 18% to about 22%, an organic acid, present from about 2% to about 15%, and in some particular embodiments present from about 6% to about 8%, and starch, present from about 40% to about 90%, and in some particular embodiments present from about 70% to about 75%.

In an exemplary embodiment, the booster composition corresponding to volumizing that includes sodium bicarbonate, an organic acid, selected from citric acid and ascorbic acid, and starch, selected from corn starch, potato starch, oat starch, rice starch, tapioca starch, sodium carboxymethyl starch.

Plumping Hair Target Attribute Booster

In the various embodiments, the plurality of water dispersible cosmeticity booster compositions includes at least one booster composition corresponding to plumping.

In some particular embodiments, the plurality of water dispersible cosmeticity booster compositions includes the booster composition corresponding to plumping that includes at least one polymeric thickener, in some embodiments comprising a polysaccharide, in some embodiments comprising a polysaccharide gellifying thickener, together with aloe, one or a combination of polyols, and optionally an active comprising a beta hydroxy acid, and water.

In some particular embodiments, the plurality of water dispersible cosmeticity booster compositions includes the booster composition corresponding to plumping that includes at least one polysaccharide gellifying thickener, present from about 0.5% to about 5%, and in some particular embodiments present from about 0.5% to about 1%, aloe, present from about 40% to about 75%, and in some particular embodiments present from about 62% to about 68%, one or a combination of polyols, each present from about 0.5% to about 20%, and in some particular embodiments each present from about 1.5% to about 10%, and optionally an active comprising a beta hydroxy acid, present from about 0.1% to about 0.3%, and in some particular embodiments present from about 0.1% to about 0.3%, and the balance water.

In an exemplary embodiment, the booster composition corresponding to plumping that includes at least one polysaccharide gellifying thickener, selected from carrageenan, aloe, one or a combination of polyols, and optionally an active comprising an organic acid, for example, salicylic acid, and water. As used variously herein, the terms plumping booster, plumping jelly, jelly booster, and plumping jelly booster all refer to a booster as described herein above.

Smoothing Hair Target Attribute Booster

In the various embodiments, the plurality of water dispersible cosmeticity booster compositions includes at least one booster composition corresponding to smoothing.

In some particular embodiments, the plurality of water dispersible cosmeticity booster compositions includes the booster composition corresponding to smoothing that includes at least one gum, and at least one hydroxy group containing compound selected from polyols, longer chain monoalcohols (C8 to C30) which include fatty alcohols, and combinations thereof, and water.

In some particular embodiments, the plurality of water dispersible cosmeticity booster compositions includes the booster composition corresponding to smoothing that includes at least one gum, present from about 0.5% to about 5%, and at least one hydroxy group containing compound selected from polyols and longer chain monoalcohols (C8 to C30), present from 0.1% to about 20%, the balance water.

In an exemplary embodiment, the booster composition corresponding to smoothing that includes at least one gum, selected from natural plant gums, selected from dehydroxanthan gum, and at least one hydroxy group containing compound selected from glycerin, caprylyl glycol, benzyl alcohol, and octyldodecanol, and water.

Repairing/Softening Hair Target Attribute Booster

In the various embodiments, the plurality of water dispersible cosmeticity booster compositions includes at least one booster composition corresponding to repairing/softening.

In some particular embodiments, the plurality of water dispersible cosmeticity booster compositions includes the booster composition corresponding to repairing butter that includes an anhydrous blend of plant/vegetable oils, and optionally at least one active comprising a vitamin.

In some particular embodiments, the plurality of water dispersible cosmeticity booster compositions includes the booster composition corresponding to repairing butter that includes an anhydrous blend of plant/vegetable oils, comprising sunflower seed oil, coconut oil, avocado oil, each present from about 0.001% to about 90%, sunflower seed oil present from about 0.001% to about 1%, and in some particular embodiments present from about 0.007% to about 0.009%, coconut oil present from about 40% to about 90%, and in some particular embodiments present from about 75% to about 80%, avocado oil present from about 5% to about 35%, and in some particular embodiments present from about 18% to about 22%, and optionally at least one active comprising a vitamin, present from about 0.00001% to about 1%, and in some particular embodiments present from about 0.00003% to about 0.00007%.

In an exemplary embodiment, the booster composition corresponding to repairing butter that includes an anhydrous blend of plant/vegetable oils, comprising sunflower seed oil, coconut oil, avocado oil, and at least one active comprising a vitamin, comprising tocopherol.

In another embodiments, the plurality of water dispersible cosmeticity booster compositions includes at least one booster composition corresponding to repairing/softening.

In some particular embodiments, the plurality of water dispersible cosmeticity booster compositions includes the booster composition corresponding to repairing/softening oil that includes an anhydrous blend comprising at least one plant oil, at least one polar emollient ester, and optionally at least one active comprising a vitamin, comprising tocopherol.

In some particular embodiments, the plurality of water dispersible cosmeticity booster compositions includes the booster composition corresponding to repairing/softening oil that includes an anhydrous blend comprising at least one plant oil, present from about 0.01% to about 3%, and in some particular embodiments present from about 1.0% to about 1.2%, the Caryocar Brasiliense oil present from about 0.01% to about 1.5%, and in some particular embodiments present from about 0.8% to about 1.2%, the jojoba oil present from about 0.1% to about 3%, and in some particular embodiments present from about 0.8% to about 1.2%, at least one polar emollient ester, present from about 50% to about 99%, and in some particular embodiments present from about 95% to about 99%, and optionally an active, present from about 0.0001% to about 1%, and in some particular embodiments present from about 0.00003% to about 0.00007%.

In an exemplary embodiment, the booster composition corresponding to repairing/softening oil that includes an anhydrous blend comprising at least one plant oil, comprising caryocar Brasiliense fruit oil and simmondsia chinesis (jojoba) seed oil, at least one ester comprising isopropyl myristate, and at least one active comprising a vitamin, comprising tocopherol.

Scenting Hair Target Attribute Booster

In the various embodiments, the plurality of water dispersible cosmeticity booster compositions includes at least one booster composition corresponding to scenting. In some particular embodiments, the plurality of water dispersible cosmeticity booster compositions includes the booster composition corresponding to scenting that includes an essentially anhydrous or anhydrous blend of at least one fragrance with at least one polar emollient. In some particular embodiments, the plurality of water dispersible cosmeticity booster compositions includes the booster composition corresponding to scenting that includes an essentially anhydrous or anhydrous blend of at least one fragrance with at least one polar emollient, comprising isopropyl palmitate, present from about 50% to about 99%, and in some particular embodiments present from about 92% to about 98%, the balance comprising at least one fragrance.

In an exemplary embodiment, the booster composition corresponding to scenting includes an essentially anhydrous or anhydrous blend of at least one fragrance, with at least one polar emollient, comprising isopropyl palm itate.

Hair Care Formulations

According to some embodiments, the system for formulating a hair care product includes in the hair product formulary schedule of hair cleansing formulation recipes that comprise base and booster compositions hereof, the schedule of recipes selected from the group consisting of:
(i) a hair cleansing formulation comprising a predetermined number of dispensed units of a paraben-free cleansing base, a predetermined number of dispensed units of a volumizing booster, and a predetermined number of dispensed units of a plumping booster;
(ii) a hair cleansing formulation comprising a predetermined number of dispensed units of a paraben-free cleansing base, and a predetermined number of dispensed units of a repairing/softening booster;
(iii) a hair cleansing formulation comprising a predetermined number of dispensed units of a paraben-free cleansing base, and a predetermined number of dispensed units of a smoothing booster;
(iv) a hair cleansing formulation comprising a predetermined number of dispensed units of a paraben-free cleansing base, and a predetermined number of dispensed units of n exfoliating booster;
(v) a hair cleansing formulation comprising a predetermined number of dispensed units of a paraben-free cleansing base, a volumizing booster, and a predetermined number of dispensed units of a plumping booster;
(vi) a hair cleansing formulation comprising a predetermined number of dispensed units of a paraben-free cleansing base, a predetermined number of dispensed units of a volumizing booster, and a predetermined number of dispensed units of a plumping booster;
(vii) a hair cleansing formulation comprising a predetermined number of dispensed units of a paraben-free cleansing base, a predetermined number of dispensed units of a smoothing booster and a predetermined number of dispensed units of a softening booster;
(viii) a hair cleansing formulation comprising a predetermined number of dispensed units of a paraben-free cleansing base, a predetermined number of dispensed units of a smoothing booster, a predetermined number of dispensed units of a softening booster, and a predetermined number of dispensed units of a volumizing booster; and
(ix) a hair cleansing formulation comprising a predetermined number of dispensed units of a paraben-free cleansing base, a predetermined number of dispensed units of a smoothing booster, and a predetermined number of dispensed units of a softening booster.

According to some embodiments, the system for formulating a hair care product includes in the hair product formulary schedule of hair conditioning formulation recipes that comprise base and booster compositions hereof, the schedule of recipes selected from the group consisting of:
(vii) a hair conditioning formulation comprising a predetermined number of dispensed units of a paraben-free conditioning base, a predetermined number of dispensed units of a repairing/softening booster, and a predetermined number of dispensed units of a plumping booster;
(viii) a hair conditioning formulation comprising a predetermined number of dispensed units of a paraben-free conditioning base and a predetermined number of dispensed units of a smoothing booster;
(ix) a hair conditioning formulation comprising a predetermined number of dispensed units of a paraben-free conditioning base and a predetermined number of dispensed units of a volumizing booster;
(x) a hair conditioning formulation comprising a predetermined number of dispensed units of a paraben-free conditioning base, a predetermined number of dispensed units of a smoothing booster, a plumping booster, and a predetermined number of dispensed units of a repairing/softening booster;
(xi) a hair conditioning formulation comprising a predetermined number of dispensed units of a paraben-free conditioning base and a repairing/softening booster;
(xii) a hair conditioning formulation comprising a predetermined number of dispensed units of a paraben-free conditioning base and a smoothing booster.

Methods

In another exemplary embodiment, a method for delivering hair care includes the steps of:
(I) Providing in any order:
   a) a hair product formulary schedule comprising a plurality of hair care formulation recipes, each hair care formulation recipe including instructions for combining one of a plurality of aqueous base compositions, and one or a combination of water dispersible cosmeticity booster compositions;
   b) an aqueous base composition disposed in a dispensing container, the dispensing container adapted to dispense a fixed unit of aqueous base composition, the aqueous base a cleansing base composition that comprises one of:
      i) a cleansing base composition; and
      ii) a conditioning base composition;
   c) a plurality of water dispersible cosmeticity booster compositions, each being readily dispersible into the base compositions and each disposed in a separate dispensing container, the plurality of water dispersible cosmeticity booster compositions comprising active ingredients corresponding to hair target attributes that include exfoliating, volumizing, plumping, smoothing, softening, repairing/softening, and scenting.
(III) Preparing a hair care formulation, including:
   d) Selecting a first hair care formulation recipe from the hair product formulary schedule;
   e) Dispensing into a mixing vessel a number of units of the aqueous base composition as set forth in the recipe;
   f) Dispensing into the mixing vessel a number of units of each of one or a combination of booster compositions as set forth in the recipe, alternately mixing after addition of each booster composition; and
   g) Completing any additional processing step as set forth in the recipe;
(IV) Delivery of hair care, including:
   h) Applying the hair care formulation to one of the scalp, the hair ends, the hair roots, and combinations thereof.

In some embodiments, the method for delivering hair care includes repeating the steps at least two times to form at least a pair of hair care formulations selected from at least one cleansing and at least one conditioning product, or at least two cleansing products, or at least two conditioning products.

Dispensing

According to some embodiments, the system for formulating a hair care product includes each of the aqueous base compositions and each of the water dispersible cosmeticity booster compositions disposed in a dispensing container that is adapted to dispense a fixed unit of the composition. According to such embodiments, each hair care formulation recipe in the hair product formulary schedule includes instructions to dispense a specific number of pre-determined fixed units of each composition into a mixing vessel. According to such embodiments, each fixed unit of composition corresponds with a predetermined amount/concentration by weight of actives. In various embodiments, the dispensing containers may be single does/fixed unit containers, for example packets, bottles, vials or tubes for solids/powderous materials, or ampules, packets, vials, tubes, or bottles for liquidous, gel, and serum materials, that may be dispensed by squeezing, pumping or other suitable means.

Ingredients Used in Base and Booster Compositions

Alkyl Polyglucosides

In accordance with the disclosure, compositions hereof may include at least one alkyl polyglucoside (also called “alkyl glucoside”). Useful alkyl polyglucosides include those having the following formula:

R¹—O—(R²O)ₙ—Z(ₓ) (I)

wherein:

R¹ is an alkyl group having 8-18 carbon atoms;

R² is an ethylene or propylene group;

Z is a saccharide group with 5-6 carbon atoms;

n is an integer ranging from 0 to 10; and

x is an integer ranging from 1 to 5.

Useful alkylpolyglucosides include lauryl glucoside, octyl glucoside, decyl glucoside, coco glucoside, caprylyl/capryl glucoside, sodium lauryl glucose carboxylate, or mixtures thereof. Typically, the alkyl polyglucoside compound is selected from the group consisting of lauryl glucoside, decyl glucoside, coco glucoside, or mixtures thereof. In some instances, decyl glucoside is particularly preferred. In some instances, coco-glucoside is particularly preferred.

In accordance with the various embodiments, the total amount of alkyl polyglucoside present in the compositions can range from about 0.1% to about 15%, or from about 0.5% to about 12%, or from about 1% to about 10%, or from about 1.5% to about 5% or any suitable combination, sub-combination, range, or sub-range thereof by weight, based on the weight of the composition. One of ordinary skill in the art, however, will appreciate that other ranges are within the scope of the invention. In accordance with some particular embodiments, at least one alkyl glucoside is present from about 1% to about 15%, and in some particular embodiments present from about 9% to about 10%.

Thus, any one of the at least one alkyl glucoside is present, by weight, based on the total weight of the composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, to about 15 weight percent, including increments and ranges therein and there between.

Cationic Polymers

In accordance with the disclosure, compositions hereof may include at least one cationic polymer.

Non-limiting examples of cationic polymers include poly(methacryloyloxyethyl trimethylammonium chloride), polyquaternium-6, polyquaternium-37 (and) propylene glycol dicaprylate/dicaprate (and) PPG-1 trideceth-6, polyquaternium-46, quaternized cellulose derivatives, polyquaternium-4, polyquaternium-10, cationic alkyl polyglycosides, cationized honey, cationic guar derivatives, polymeric dimethyl diallyl ammonium salts and copolymers thereof with esters and amides of acrylic acid and methacrylic acid, copolymers of vinyl pyrrolidone with quaternized derivatives of dialkylaminoalkyl acrylate and methacrylate, vinyl pyrrolidone-vinyl imidazolium methochloride copolymers, quaternized polyvinyl alcohol, polyquaternium-2, polyquaternium-7, polyquaternium-17, polyquaternium-18, polyquaternium-24, polyquaternium-27, and a mixture thereof. In some instances, the one or more cationic polymers may be selected from the group consisting of polyquaternium-4, polyquaternium-10, cationic guar derivatives, and a mixture thereof.

The cationic polymers can be a monoalkyl quaternary amine, such as stearyltrimonium chloride, soyatrimonium chloride or coco-ethyldimonium ethosulfate. Other suitable cationic polymers include, but are not limited to, behentrimonium chloride, dialkyl quaternary amines, such as dicetyldimonium chloride, dicocodimethyl ammonium chloride or distearyldimethyl ammonium chloride; and polyquaternium compounds, such as Polyquaternium-6, Polyquaternium-22 or Polyquaternium-5.

For example, cationic polymers may be chosen from polyquaterium-10 (also called quaternized polyhydroxyethyl cellulose), cetrimonium chloride (also called cetyl trimethyl ammonium chloride, CTAC), behentrimonium chloride (also known as docosyl trimethyl ammonium chloride), behentrimonium methosulfate, steartrimonium chloride, stearalkonium chloride, dicetyldimonium chloride, guar hydroxypropyltrimonium chloride, hydroxypropyl guar hydroxypropyltrimonium chloride, cocotrimonium methosulfate, olealkonium chloride, steartrimonium chloride, babassuamidopropalkonium chloride, brassicamidopropyl dimethylamine, Quaternium-91, Salcare/PQ-37, Quaternium-22, Quaternium-87, Polyquaternium-4, Polyquaternium-6, Polyquaternium-11, Polyquaternium-44, Polyquaternium-67, amodimethicone, lauryl betaine, Polyacrylate-1 Crosspolymer, steardimonium hydroxypropyl hydrolyzed wheat protein, behenamidopropyl PG-dimonium chloride, lauryldimonium hydroxypropyl hydrolyzed soy protein, am inopropyl dimethicone, Quaterium-8, and dilinoleamidopropyl dimethylamine dimethicone PEG-7 phosphate.

In some instances, the cationic polymers are cationic conditioning polymers. Examples of cationic conditioning polymers that can be used include, without limitation, cationic cellulose, cationic proteins, and cationic polymers. The cationic polymers can have a vinyl group backbone of amino and/or quaternary ammonium monomers. Cationic amino and quaternary ammonium monomers include, without limitation, dialkylamino alkylmethacrylate, monoalkylaminoalkyl acrylate, monoalkylaminoalkyl methacrylate, trialkyl methacryoloxyalkyl ammonium salt, trialkyl acryloxyalkyl ammonium salts, diallyl quaternary ammonium salts, vinyl compounds substituted with dialkyl aminoalkyl acrylate, and vinyl quaternary ammonium monomers having cyclic cationic nitrogen containing rings such as pyridinium, imidazolium, or quaternized pyrrolidine. Other examples of cationic conditioning polymers that can be used include, without limitation, hydroxypropyltrimonium honey, cocodimonium silk amino acids, cocodimonium hydroxypropyl hydrolyzed wheat or silk protein, polyquaternium-5, polyquaternium-11, polyquaternium-2, polyquaternium-4, polyquaternium-6, polyquaternium-7, polyquaternium-14, polyquaternium-16, polyquaternium-22, polyquaternium-10, and guar hydroxypropyltrimonium chloride.

In some cases, quaternized polymeric cationic polymers are particularly useful. Some cationic polymers are quaternary nitrogen polymers prepared by the polymerization of a dialkyldiallylammonium salt or copolymer thereof in which the alkyl group contains 1 to about 18 carbon atoms, and more preferably where the alkyl group is methyl or ethyl. Details concerning the preparation of these polymers can be found in U.S. Pat. Nos. 3,288,770, 3,412,019 and 4,772,462, incorporated herein by reference. For example, cationic homopolymers and copolymers of polydiallyldimethylammonium chloride are available in aqueous compositions sold under the trademark MERQUAT by the Calgon Corporation, subsidiary of Merck & Co., Pittsburgh, Pa. The homopolymer, which is named Polyquaternium-6 is sold under the trademark MERQUAT-100 and is described as having a weight average molecular weight of approximately 100,000. A copolymer reaction product of dimethyldiallylammonium chloride with acrylamide monomers is named Polyquaternium-7 is described as having a weight average molecular weight of approximately 500,000 and is sold under the trademark MERQUAT-550. Another copolymer reaction product of dimethyldiallylammonium chloride with acrylic acids having a weight average molecular weight from about 50,000 to about 10,000,000 has the name Polyquaternium-22 and is sold under the trademark MERQUAT-280. Polyquaternium-6 is particularly preferred.

Other polymeric conditioners include cationic copolymers of methylvinylimidazolium chloride and vinyl pyrrolidone, sold commercially by BASF Aktiengesellschaft, West Germany under the trademark LUVIQUAT at three comonomer ratios, namely at ratios of 95/5, 50/50 and 30/70 methylvinylimidazolium chloride to polyvinylpyrrolidone. These copolymers at all three comonomer ratios have the name Polyquaternium 16. Polymeric conditioners also include cationic cellulosic polymers of hydroxyethyl cellulose reacted with epichlorohydrin and quaternized with trimethylamine, sold under the trademark POLYMER JR in various viscosity grades and molecular sizes by Union Carbide Corporation, Danbury, Conn. These series of polymers are named Polyquaternium 10. Also useful are quaternized copolymers of hydroxyethylcellulose and dimethyldimethylammonium chloride, having the name Polyquaternium-4, sold in varying molecular weights under the trademark CELQUAT by National Starch and Chemical Corporation, Bridgewater, N.J.

Smaller molecule cationic non-polymeric conditioning agents can also be utilized herein. Exemplary small-molecule conditioning agents can include monofunctional or difunctional quaternary ammonium compounds, such as stearyldimethylbenzylammonium chloride, dimethyldi-(hydrogenated tallow)ammonium chloride, and the like. Non-polymeric conditioning agents can also include the quaternary ammonium salts of gluconamide derivatives, such as gamma-gluconamidopropyldimethyl-2-hydroxyethyl-ammonium chloride and minkamidopropyldimethyl-2-hydroxyethylammonium chloride identified respectively by the names Quaternium 22 and Quaternium 26. Details for the preparation of these materials are found in U.S. Pat. Nos. 3,766,267 and 4,012,398, respectively, and the materials are sold under the trademark CERAPHYL by Van Dyk & Co., Belleville, N.J. Also useful are bis-quaternary ammonium compounds which are dimers, such as 2-hydroxy propylene-bis-1,3-(dimethylstearyl ammonium chloride, designated the name, Hydroxypropyl Bisstearyldimonium chloride. The preparation of these and other bis-quat materials is described in U.S. Pat. No. 4,734,277, and such materials are sold under the trademark JORDAQUAT DIMER by Jordan Chemical Company, Folcroft, Pa.

Exemplary unquaternized polymers having tertiary amino nitrogen groups that become quaternized when protonated can include water-soluble proteinaceous quaternary ammonium compounds. Cocodimonium hydrolyzed animal protein, for example, is the name for a chemically modified quaternary ammonium derivative of hydrolyzed collagen protein having from about 12 to about 18 carbons in at least one aliphatic alkyl group, a weight average molecular weight from about 2500 to about 12,000, and an isoionic point in a range from about 9.5 to about 11.5. This material and structurally related materials are sold under the trademarks CROQUAT and CROTEIN by Croda, Inc., New York, N.Y.

In some particular embodiments, the at least one cationic polymer selected from Polyquaternium-6, polyquaternium-10, polyquaternium-4, and guar hydroxypropyltrimonium chloride, hydroxypropyl guar hydroxypropyltrimonium chloride, and combinations thereof.

In accordance with the various embodiments, the amount of each of the at least one cationic polymer is from about 0.1% to about 10%, or from about 0.1% to about 8%, or from about 0.2% to about 7%, or from about 1% to about 5%, or from about 1.5% to about 3% or any suitable combination, sub-combination, range, or sub-range thereof by weight, based on the weight of the composition. One of ordinary skill in the art, however, will appreciate that other ranges are within the scope of the invention.

In accordance with the various embodiments, the total amount of cationic polymer present in the compositions can range from about 0.01% to about 15%, or from about 0.5% to about 12%, or from about 1% to about 10%, or from about 1.5% to about 5%, or from about 0.01% to about 3%, or from about 0.05% to about 0.15%, or any suitable combination, sub-combination, range, or sub-range thereof by weight, based on the weight of the composition. One of ordinary skill in the art, however, will appreciate that other ranges are within the scope of the invention. In some particular embodiments, at least one cationic polymer present from about 0.01% to about 3%, and in some particular embodiments present from about 0.05% to about 0.15%

Thus, any one of the at least one cationic polymer is present, by weight, based on the total weight of the composition, from about 0.10, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14 to about 15 weight percent, including increments and ranges therein and there between.

Cationic Surfactant

In accordance with the disclosure, compositions hereof may include at least one cationic surfactant. The term “cationic surfactant” means a surfactant that may be positively charged when it is contained in the compositions according to the disclosure. This surfactant may bear one or more positive permanent charges or may contain one or more functional groups that are cationizable in the composition according to the disclosure. Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In some embodiments, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some embodiments, the cationic surfactant comprises cetrimonium chloride, behentrimonium chloride, and mixtures thereof. Behentrimonium Chloride, also described by the technical names that include 1-Docosanaminium, N,N,N-Trimethyl-, Chloride, and N,N,N-Trimethyl-1-Docosanaminium Chloride, is the quaternary ammonium salt that conforms to the formula:

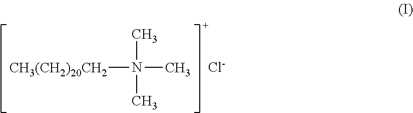

In accordance with some embodiments, the amount of each of the at least one cationic surfactant is from about 0.5% to about 10%, or from about 0.5% to about 8%, or from about 1% to about 5%, or from about 1.5% to about 3% or any suitable combination, sub-combination, range, or sub-range thereof by weight, based on the weight of the composition. One of ordinary skill in the art, however, will appreciate that other ranges are within the scope of the invention. In some particular embodiments, the at least one cationic surfactant, including cationizable surfactants together with an acid neutralizer, is present from about 0.5% to about 5%, and in some particular embodiments is present from about 2.25% to about 2.75%, and when present, an acid neutralizer present from about 0.25% to about 0.5%.

In accordance with the various embodiments, the total amount of cationic surfactant present in the compositions can range from about 0.5% to about 20%, or from about 0.5% to about 10%, or from about 0.5% to about 5%, or from about 1% to about 3% or any suitable combination, sub-combination, range, or sub-range thereof by weight, based on the weight of the composition. One of ordinary skill in the art, however, will appreciate that other ranges are within the scope of the invention.

Thus, any one of the at least one cationic surfactant is present, by weight, based on the total weight of the composition, from about 0.5, 0.6, 0.7, 0.8, 0.9, 1, 2, 3, 4, 5, 6, 7, 8, 9, to about 10 weight percent, including increments and ranges therein and there between.

Non-limiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and mixtures thereof.

In some cases, it is useful to use salts such as chloride salts of the quaternary ammonium compounds.

The fatty amines generally comprise at least one C₈-C₃₀ hydrocarbon-based chain.

A. Examples of quaternary ammonium salts that may especially be mentioned include: those corresponding to the general formula (III) below:

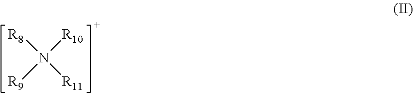

in which the groups R₈ to R₁₁ which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and, in some embodiments, from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

B. a quaternary ammonium salt of imidazoline, such as, for example, those of formula (IV) below:

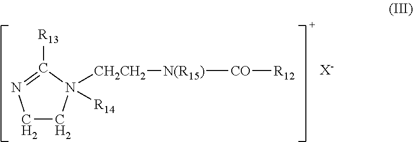

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻is an anion chosen from the group of halides, phosphates, acetates, lactates, alkyl sulfates, alkyl- or alkylaryl-sulfonates in which the alkyl and aryl groups, in some embodiments, comprise, respectively, from 1 to 20 carbon atoms and from 6 to 30 carbon atoms. R₁₂ and R₁₃ in some embodiments, denote a mixture of alkenyl or alkyl groups containing from 12 to 21 carbon atoms, derived for example from tallow fatty acids, R₁₄ in some embodiments, denotes a methyl group, and R₁₅ in some embodiments, denotes a hydrogen atom. Such a product is sold, for example, under the name REWOQUAT W 75 by the company Rewo;

C. a quaternary diammonium or triammonium salt, in particular of formula (V):

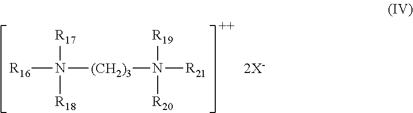

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N⁻(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75),

D. Cationic/cationizable surfactants, for example of the general structure R4-A-R5B

wherein R4 is a saturated or unsaturated, straight or branched alkyl chain with 8 to 24 C atoms, R5 is a straight or branched alkyl chain with 1 to 4 C atoms, A is selected from:

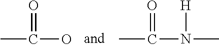

and B is selected from

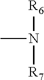

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms,

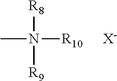

R₈ and R₉ are the same or different, an alkyl chain with 1 to 4 C atoms, hydroxyl alkyl chain with 1 to 4 C atoms and di hydroxyl alkyl chain with 2 to 4 C atoms, R.sub. 10 is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight or branched alkyl chain with 10 to 24C atoms, in some embodiments, 12 to 22 C atoms and R₅ is straight or branched alkyl group with 1 to 4 C atoms, and A, B. R₆ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine. lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine. behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine. palmitoylpropyl buylamine. palmitoylpropyl dipropylamine, palmitoylpropyl propylamine, palmitoylpropyl dihydroxyethylamine, palmitoylpropyl hydroxyethylamine. palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine. myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine. behenylpropyl dibutylamine. behenylpropyl butylamine, behenylpropyl dipropylamine, behenylpropyl propylamine, behenylpropyl dihydroxyethylamine, behenylpropyl hydroxyethylamine. behenylpropyl dihydroxypropylamine, behenylpropyl hydroxypropylamine, dipalm itoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants or amphiphilic surfactants may be chosen from fatty alkylamines, in some embodiments, fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dim ethylamine, myristamidopropyl dim ethylamine, dilinoleamidopropyl dim ethylamine, and palmitamidopropyl dim ethylamine.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and mixtures thereof. In particular, lactic acid or tartaric acid or mixtures thereof are useful, especially in combination with fatty dimethylamines such as, for example, stearamidopropyl dimethylamine.

Fatty Alcohols

In accordance with the disclosure, compositions hereof may include at least one fatty alcohol.

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. In some instances, it is preferable that the hair treatment compositions include at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In particular, it is possible to mention, alone or as a mixture: lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol).

Preferably, the solid fatty alcohol is chosen from cetyl alcohol, stearyl alcohol, behenyl alcohol and mixtures thereof such as cetylstearyl or cetearyl alcohol.

The liquid fatty alcohols, in particular those containing C₁₀-C₃₄, preferably have branched carbon chains and/or have one or more, preferably 1 to 3 double bonds. They are preferably branched and/or unsaturated (C═C double bond) and contain from 12 to 40 carbon atoms.

The liquid fatty alcohols may be represented by: R—OH, wherein R denotes a C₁₂-C₂₄ branched alkyl group or an alkenyl group (comprising at least one C₁₂-C₂₄ double bond C═C), R being optionally substituted by a or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

In some instances, the hair treatment compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the hair treatment compositions preferably include cetearyl alcohol.

In accordance with the various embodiments, when present, the amount of each of the at least one fatty alcohol is from about 0.1% to about 10%, or from about 0.1% to about 8%, or from about 0.2% to about 7%, or from about 1% to about 5%, or from about 1.5% to about 3% or any suitable combination, sub-combination, range, or sub-range thereof by weight, based on the weight of the composition. One of ordinary skill in the art, however, will appreciate that other ranges are within the scope of the invention.

In some particular embodiments, at least one fatty alcohol is present from about 0.5% to about 15%, and in some particular embodiments present from about 2% to about 4%.

In accordance with the various embodiments, the total amount of fatty alcohol present in the compositions can range from about 0.1% to about 15%, or from about 0.5% to about 12%, or from about 1% to about 10%, or from about 1.5% to about 5% or any suitable combination, sub-combination, range, or sub-range thereof by weight, based on the weight of the composition. One of ordinary skill in the art, however, will appreciate that other ranges are within the scope of the invention.

Thus, any one of the at least one fatty alcohol is present, by weight, based on the total weight of the composition, from about 0, 0.2, 0.3, 0.4, 0.5, 0.6, 0.7, 0.8, 0.9, 1, 2, 3, 4, 5, 6, 7, 8, 9, to about 10 weight percent, including increments and ranges therein and there between.

Polar Emollients

In accordance with the disclosure, compositions hereof may include at least one polar emollient. Emollients are oil-phase ingredients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol to about 350 g/mol.

In some embodiments, suitable polar emollients that may be used according to the disclosure include those derived from C₁₂-050 fatty acids, preferably C₁₆-C₂₂ saturated fatty acids, and monohydric alcohols. In some embodiments, such esters may be chosen from isopropyl myristate, methyl palmitate, isopropyl laurate, isopropyl palm itate, ethylhexyl palm itate, ethylhexyl laurate, ethylhexyl oleate, ethylhexyl isononoanoat, myristyl myristate, 2-ethylhexyl caprate/caprylate (or octyl caprate/caprylate), 2-ethylhexyl palm itate, isostearyl neopentanoate, isononyl isononanoate, hexyl laurate, esters of lactic acid and of fatty alcohols comprising 12 or 13 carbon atoms, dicaprylyl carbonate and their mixtures.

In some embodiments, the one or more polar emollient comprises one of isopropyl myristate, dicaprylyl ether, ehylhexyl palmitate, isopropyl palmitate, cetearyl ethylhexanoate, isononyl isononanoate, isopropryl isostearate, diisopropyl sebacate, coco caprylate/caprate, diisopropyl adipate, and combinations thereof. In some particular embodiments the one or more polar emollient includes isopropyl palmitate, isopropyl myristate, and combinations thereof. In some embodiments the composition comprises two or more polar emollients. In some such embodiments, the polar emollient comprises isopropyl palm itate, isopropyl myristate, and combinations thereof and one or more additional polar emollients.

The amount of each of the at least one polar emollient is present in the composition in an range of from about 1% to about 99% by weight, or from about 10% to about 90% by weight, or from about 25% to about 80% or any suitable combination, sub-combination, range, or sub-range thereof by weight, based on the weight of the composition. One of ordinary skill in the art, however, will appreciate that other ranges are within the scope of the invention. In some particular embodiments, the at least one polar emollient is present from about 50% to about 99%.

Thus, each of the at least one polar emollient in the composition may be present by weight, based on the total weight of the composition, from about 1, 2, 3, 4, 5, 6, 7, 8, 9, 10, 15, 20, 25, 30, 35, 40, 45, 50, 55, 60, 65, 70, 75, 80, 85, 90, 95 to about 99 percent, including increments and ranges therein and there between.

Polymeric Thickener

In accordance with the disclosure, compositions hereof may include one or more polymeric thickeners.

In some embodiments, the one or more thickener may be selected from one or more of natural gums and synthetic polymers, for example starches (corn, rice, tapioca, potato), gums (xanthan, carrageenan, gellan, sclerotium, tarabiotech fermentation). In some particular embodiments, the thickener may be selected from acrylates/C₁₀-30 alkyl acrylate crosspolymer, carbomer, xanthan gum, hydroxypropyl guar, ceratonia siliqua (carob) gum, ammonium polyacryloyldimethyl taurate, ammonium acryloyldimethyltaurate/steareth-25 methacrylate crosspolymer, and polyacrylate crosspolymer-6.

In some embodiments, the polymeric thickener is one of carbomer, acrylates/C₁₀-30 alkyl acrylate crosspolymer, polyacrylate crosspolymer-6, microcrystalline cellulose (and) cellulose gum, xanthan gum, sodium carboxymethyl starch, sclerotium gum (and) xanthan gum, xanthan gum (and) ceratonia siliqua (carob) gum (50/50), dehydroxanthan gum, hydroxypropyl starch phosphate, sclerotium gum (and) xanthan gum (75/25), sclerotium gum, xanthan gum (and) sclerotium gum (and) Lecithin (and) pullulan, and combinations thereof.

In some embodiments the composition comprises two or more polymeric thickeners. In some such embodiments, the polymeric thickener comprises one or both of acrylates/C₁₀-30 alkyl acrylate crosspolymer and carbomer, and optionally one or more additional polymeric thickeners.

The amount of each of the at least one polymeric thickener, when present, is present in the composition in a range of from about 0.01% to about 2%, or from about 0.01% to about 1.5%, or from about 0.3% to about 1.2%, or any suitable combination, sub-combination, range, or sub-range thereof by weight, based on the weight of the composition. One of ordinary skill in the art, however, will appreciate that other ranges are within the scope of the invention. In some particular embodiments, the polymeric thickener is present from about 0.5% to about 5%.

In some embodiments, when present, the total amount of polymeric thickener in the composition is present from about 0.01% to about 5%, or from about 0.02% to about 2%, or from about 0.03% to about 1.5%, or from about 0.1% to about 0.2%, or any suitable combination, sub-combination, range, or sub-range thereof by weight, based on the weight of the composition. One of ordinary skill in the art, however, will appreciate that other ranges are within the scope of the invention.

Thus, one or more polymeric thickener, when present, is present by weight, based on the total weight of the composition, from about 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5 to about 5.0 percent, including increments and ranges therein and there between.

Polysaccharide

In accordance with the disclosure, compositions hereof may include at least one polysaccharide.

In general, the polysaccharide may be chosen from polysaccharides isolated from algae, polysaccharides produced by microorganisms, and polysaccharides from higher plants, such as homogeneous polysaccharides. In particular, the polysaccharides may be chosen from fructans, galactans, pullulan, and derivatives thereof. In other examples, the polysaccharides may comprise methylcelluloses, hydroxyalkylcelluloses, ethylhydroxyethylcelluloses and carboxymethylcelluloses, mannans, xylans, lignins, arabans, galacturonans, alginate-based compounds, chitin, chitosans, glucuronoxylans, arabinoxylans, xyloglucans, glucomannans, pectic acids and pectins, arabinogalactans, agars, glycosaminoglycans, gum arabics, tragacanth gums, ghatti gums, karaya gums, locust bean gums, galactomannans such as guar gums and nonionic derivatives thereof, in particular hydroxypropyl guar, and ionic derivatives thereof, biopolysaccharide gums of microbial origin, in particular scleroglucan or xanthan gums, mucopolysaccharides, and in particular chondroitin sulfates, and mixtures thereof.

In some embodiments, the at least one polysaccharide may be chosen from fructans, galactans, pullulan, derivatives thereof and combinations thereof. According to the various embodiments, the at least one polysaccharide may be chemically modified, especially with urea or urethane groups, or by a hydrolysis, oxidation, esterification, etherification, sulfatation, phosphatation, amination, am idation or alkylation reaction, or by several of these modifications. The derivatives obtained may be anionic, cationic, amphoteric or nonionic.

Polysaccharides Isolated from Algae: Fructosans

In some particular embodiments, the polysaccharide according to the invention, when present, may especially be a fructosan chosen from inulin and derivatives thereof (especially dicarboxy and carboxymethyl inulins). Fructans or fructosans are oligosaccharides or polysaccharides comprising a sequence of anhydrofructose units optionally combined with several saccharide residues other than fructose. Fructans may be linear or branched. Fructans may be products obtained directly from a vegetable or microbial source or alternatively products whose chain length has been modified (increased or decreased) by fractionation, synthesis or hydrolysis, in particular enzymatic. Fructans generally have a degree of polymerization from 2 to about 1000 and in some embodiments, from 2 to about 60.

Three groups of fructans are distinguished. The first group corresponds to products whose fructose units are for the most part linked via 13(2,1) bonds. These are essentially linear fructans such as inulins. The second group also corresponds to linear fructoses, but the fructose units are essentially linked via 13(2,6) bonds. These products are levans. The third group corresponds to mixed fructans, i.e. fructans containing 13(2,6) and β(2,1) sequences. These are essentially branched fructans, such as graminans. Inulin is also referred to technically as Alantin, Fructosane, Synantherin, and Synanthrin

Inulin may be obtained, for example, from chicory, dahlia or Jerusalem artichoke, in some embodiments, from chicory. In particular, the polysaccharide, especially the inulin, has a degree of polymerization from 2 to about 1000 and, in some embodiments, from 2 to about 60, and a degree of substitution of less than 2 on the basis of one fructose unit. The inulin used for this invention is represented, for example, by the products available under the tradename INUTEC H25P (CREACHEM), also sold under the name Beneo™ Inulin by the company Orafti, and under the name Frutafit™ by the company Sensus.

Polysaccharides Isolated from Algae: Galactans

In some particular embodiments, the polysaccharide according to the invention, when present, may be a galactan chosen especially from agar and carrageenans. Galactans of agar type are galactose polysaccharides contained in the cell wall of some of these species of red algae (Rhodophyceae) belonging to the Gigartinaceae, Hypneaceae, Furcellariaceae and Polyideaceae families. They are formed from a polymer group in which the base backbone is a 13(1,3) D-galactopyranose and α(1,4) L 3-6 anhydrogalactose chain, these units repeating regularly and alternately. The differences within the agar family are due to the presence or absence of methyl or carboxyethyl solvated groups. These hybrid structures are generally present in variable percentage, depending on the species of algae and the season of harvest.

They are generally obtained by hot aqueous extraction from natural strains of the said algae. These linear polymers, formed by disaccharide units, are composed of two D-galactopyranose units alternately linked via α(1,3) and β(1,4) bonds. These are highly sulfated polysaccharides (20-50%) and the α-D-galactopyranosyl residues may be in 3,6-anhydro form. According to the number and position of the ester sulfate groups on the repeat disaccharide of the molecule, several types of carrageenan are distinguished, namely: kappa-carrageenans, which bear one ester sulfate group, iota-carrageenans which bear two ester sulfate groups, and lambda-carrageenans which bear three ester sulfate groups.

Carrageenans are composed essentially of potassium, sodium, magnesium, triethanolamine and/or calcium salts and of ester sulfates of polysaccharides. Carrageenans are sold especially by the company SEPPIC under the name Solagum™, by the company Gelymar under the names Carragel™, Carralact™ and Carrasol™, by the company Cargill under the names Satiagel™ and Satiagum™, and by the company CP-Kelco under the names Genulacta™, Genugel™ and Genuvisco™.

Polysaccharides Produced by Microorganisms: Pullulan

In some particular embodiments, the polysaccharide according to the invention, when present, may be pullulan. Pullulan is a polysaccharide consisting of maltotriose units, known under the name α(1,4)-α(1,6)-glucan. Three glucose units in maltotriose are connected via an α(1,4) glycosidic bond, whereas the consecutive maltotriose units are connected to each other via an α(1,6) glycosidic bond. Pullulan is produced, for example, under the reference Pullulan PF 20 by the company Hayashibara in Japan.

In general, the compounds of this type that may be used in the present invention are chosen from those described especially in Kirk-Othmer's Encyclopedia of Chemical Technology, Third Edition, 1982, volume 3, pp. 896-900, and volume 15, pp. 439-458, in Polymers in Nature by E. A. McGregor and C. T. Greenwood, published by John Wiley & Sons, Chapter 6, pp. 240-328, 1980, in the publication by Robert L. Davidson entitled Handbook of Water-soluble Gums and Resins published by McGraw-Hill Book Company (1980) and in Industrial Gums-Polysaccharides and their Derivatives, edited by Roy L. Whistler, Second Edition, published by Academic Press Inc.

In accordance with the various embodiments, when present, the amount of each of the at least one polysaccharide present in the compositions can range from about 0.5% to about 10%, or from about 0.5% to about 7%, or from about 1% to about 3.5% or any suitable combination, sub-combination, range, or sub-range thereof by weight, based on the weight of the composition. One of ordinary skill in the art, however, will appreciate that other ranges are within the scope of the invention.

In accordance with the various embodiments, when present, the total amount of polysaccharide present in the compositions can range from about 0.5% to about 20%, or from about 0.5% to about 10%, or from about 0.5% to about 5%, or from about 1% to about 3% or any suitable combination, sub-combination, range, or sub-range thereof by weight, based on the weight of the composition. One of ordinary skill in the art, however, will appreciate that other ranges are within the scope of the invention.

Thus, any one of the at least one polysaccharide, when present, is present, by weight, based on the total weight of the composition, from about 0.5, 0.6, 0.7, 0.8, 0.9, 1, 2, 3, 4, 5, 6, 7, 8, 9, to about 10 weight percent, including increments and ranges therein and there between.

Water

In accordance with the various embodiments water is present in the water containing compositions and the formulations and regimens hereof in total amounts of from about 15%, or from about 20%, or from about 25%, or from about 60%, or from about 65%, or from about 70%, or from about 75%, or from about 80%, or from about 85%, or from about 90%, and up to about 99%, or up to about 95%, or up to about 90%, or up to about 85%, or up to about 80%, or up to about 75%, or up to about 70%, or up to about 20%. For example, water is present in amounts ranging from about 15%, to about 25%, to about 25%, to about 35%, to about 40%, to about 50%, to about 60%, 70%, 75%, 80%, 85%, 90%, 95%, or 99% by weight, or any suitable combination, sub-combination, range, or sub-range thereof by weight, based on the weight of the composition. One of ordinary skill in the art, however, will appreciate that other ranges are within the scope of the invention.

Thus, any water is present, by weight, based on the total weight of the composition, from about 15, 20, 25, 30, 35, 40, 45, 50, 55, 56, 57, 58, 59, 60, 61, 62, 63, 64, 65, 66, 67, 68, 69, 70, 71, 72, 73, 74, 75,76, 77, 78, 79, 80, 85, 90, 95, to about 99 weight percent, including increments and ranges therein and there between.

Water-Soluble Solvents

In accordance with some embodiments, the hair treatment compositions, formulations and regimens hereof may include at least one water-soluble solvent, providing such additive does not adversely affect the effectiveness of actives directed to hair target attributes. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C₁-C₃₀, C₁-C₁₅, C₁-C₁₀, or C₁-C₄ alcohols), organic solvents, polyols, glycols, or mixtures thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylne glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In some cases, the water-soluble solvent may be selected from the group consisting of one or more glycols, C₁-4 alcohols, glycerin, or mixtures thereof, for example, glycerin, ethylhexylglycerin, and mixtures thereof.

In accordance with the various embodiments total amount of the at least one water-soluble solvent, when present, may vary, but is typically from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the composition. One of ordinary skill in the art, however, will appreciate that other ranges are within the scope of the invention.

Thus, any one water-soluble solvent, when present, is present, by weight, based on the total weight of the composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

Additional Ingredients

In accordance with some embodiments, the hair treatment compositions, formulations and regimens hereof may include at least one additional miscellaneous ingredient or additives such as organic acids, for example, alpha and beta hydroxy acids, antioxidants, for example vitamins, polyphenols and combinations thereof, essential oils, plant or fruit oils, humectants including polyols, preservatives, chelating agents, UV filters, pH adjusters, pigments, colorants, anti-dandruff, seborrheic agents, fragrances, and other skin actives, providing such additive does not adversely affect the effectiveness of actives directed to hair target attributes. In some particular embodiments, a composition hereof may include one or more of at least one active comprising a vitamin, for example tocopherol, a preservative, a polyol, a beta hydroxy acid, and combinations thereof.

In accordance with the various embodiments, when present, an additive is present from about 0.00001% to about 20%, or from about 0.0001% to about 20%, or from about 0.001% to about 20%, or from about 0.01% to about 20%, or from about 0.05% to about 15%, or from about 0.1% to about 12%, or from about 1% to about 10%, or from about 1.5% to about 5% or any suitable combination, sub-combination, range, or sub-range thereof by weight, based on the weight of the composition. One of ordinary skill in the art, however, will appreciate that other ranges are within the scope of the invention.

Thus, when present, an additive is present, by weight, based on the total weight of the composition, from about 0.00001, 0.00005, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 weight percent, including increments and ranges therein and there between.

In accordance with some embodiments, the hair treatment compositions, formulations and regimens hereof may include at least one preservative. Suitable preservatives include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, benzoic acid, chlorhexidine digluconate, ethylenediamine-tetraacetic acid (EDTA), potassium sorbate, and/or grapefruit seed extract, or a mixture thereof. Other preservatives are known in the cosmetics industries and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, Vitamin E (tocopherol), and a mixture thereof. In some cases, the hair-treatment compositions may include one or more preservatives selected from the group consisting of sodium benzoate, benzoic acid, chlorhexidine digluconate, chlorhexidine dihydrochloride, salicylic acid, phenoxyethanol, methyl paraben, and a mixture thereof.

In accordance with the various embodiments, when present, an additive in the form of one or more preservative is from about 0.1% to about 5%, or from about 0.1% to about 5%, or from about 0.2% to about 5%, or from about 1.5% to about 3% or any suitable combination, sub-combination, range, or sub-range thereof by weight, based on the weight of the composition. One of ordinary skill in the art, however, will appreciate that other ranges are within the scope of the invention.

The following examples are intended to further illustrate the present disclosure. They are not intended to limit the disclosure in any way. Unless otherwise indicated, all parts are by weight.

### EXAMPLES

In the following tables, all amounts are shown as weight percent based on weight of the composition.

### Example 1: Cleanser Base (Cleansing Liquid Base)

TABLE 1
CLEANSING LIQUID BASE

| INGREDIENT INCI | EX 1: CLEANSING BASE |
|---|---|
| ALKYL POLYGLUCOSIDE: COCO- | 9.36 |
| GLUCOSIDE AND/OR DECYL | |
| GLUCOSIDE AND/OR LAURYL | |
| GLUCOSIDE | |
| SALICYLIC ACID | 0.5 |
| CATIONIC POLYMER: | 0.1 |
| HYDROXYPROPYL GUAR | |
| HYDROXYPROPYLTRIMONIUM | |
| CHLORIDE AND/OR GUAR | |
| HYDROXYPROPYLTRIMONIUM | |
| CHLORIDE | |
| WATER | Q.S. 100 |

### Example 2: Conditioning Base (Conditioning Milk)

TABLE 2
CONDITIONING BASE

| | EX 2: |
|---|---|
| INGREDIENT INCI | CONDITIONING BASE |
| CATIONIC SURFACTANT: | 2.5 |
| BRASSICAMIDOPROPYL | |
| DIMETHYLAMINE AND/OR | |
| BEHENTRIMONIUM CHLORIDE AND/OR | |
| CETRIMONIUM CHLORIDE AND/OR | |
| QUATERNIUM-87 | |
| CAPRYLYL GLYCOL | 0.3 |
| FATTY ALCOHOLS: CETEARYL | 3 |
| ALCOHOL AND/OR CETYL ALCOHOL, | |
| AD/OR STEARYL ALCOHOL, MYRISTYL | |
| ALCOHOL, LAURYL ALCOHOL, | |
| BEHENYL ALCOHOL | |
| SALICYLIC ACID | 0.2 |
| WATER | Q.S. 100 |
| OPTIONAL ADDITIVES: VITAMINS, | <0.5 |
| PLANT OILS, NEUTRALIZERS, FOR | |
| EXAMPLE, GLYCINE SOJA (SOYBEAN) | |
| OIL, TARTARIC ACID | |

### Example 3: Smooth Booster

TABLE 3
SMOOTH BOOSTER

| | EX 3: |
|---|---|
| | SMOOTH |
| INGREDIENT INCI | BOOSTER |
| CARAMEL | 0.05 |
| OCTYLDODECANOL AND BENZYL ALCOHOL | 3 |
| GUMS: DEHYDROXANTHAN GUM AND/OR | 1.5 |
| MICROCRYSTALLINE GUM AND/OR CELLULOSE | |
| GUM AND/OR HYDROXYETHYL CELLULOSE AND/OR | |
| HYDROXYPROPYL GUAR GUM AND/OR XANTHAN | |
| GUM AND/OR SODIUM CARBOXYMETHYL | |
| CELLULOSE GUM AND/OR SCLEROTIUM GUM | |
| AND/OR XANTHAN GUM | |
| WATER | Q.S. 100 |
| POLYOLS: GLYCERIN AND/OR PROPYLENE GLYCOL | 4.3 |
| AND/OR BUTYLENE GLYCOL AND/OR CAPRYLYL | |
| GLYCOL | |

### Example 4: Plumping Jelly Booster-Paraben Free

TABLE 4
JELLY BOOSTER

| INCI US | 1129934MX8 |
|---|---|
| CARRAGEENAN | 1 |
| ALOE BARBADENSIS LEAF JUICE | 64.8245 |
| POTASSIUM SORBATE | 0.065 |
| SODIUM BENZOATE | 0.5325 |
| GLYCERIN | 7 |
| PROPYLENE GLYCOL | 1.5 |
| SALICYLIC ACID | 0.2 |
| WATER | 24.8 |
| CITRIC ACID | 0.078 |

### Example 5: Repairing/softening Butter (Paraben Free)

TABLE 5
REPAIRING/SOFTENING BUTTER

| INCI US | 1129935MX4 |
|---|---|
| TOCOPHEROL | 0.00005 |
| ANNATTO | 0.00125 |
| HELIANTHUS ANNUUS | 0.0087 |
| (SUNFLOWER) SEED OIL | |
| COCOS NUCIFERA (COCONUT) OIL | 79.99 |
| PERSEA GRATISSIMA (AVOCADO) OIL | 20 |

### Example 6: Volumizing Starch (Paraben Free)

TABLE 6
VOLUMIZING STARCH

| INCI US | 1129936MX1 |
|---|---|
| SODIUM BICARBONATE | 20 |
| CITRIC ACID | 7 |
| ZEA MAYS (CORN) STARCH | 73 |

### Example 7: Exfoliate Grains Booster (Paraben Free)

TABLE 7
EXFOLIATE GRAINS BOOSTER

| INGREDIENT INCI | EX 7: EXFOLIATE GRAINS BOOSTER |
|---|---|
| ARGANIA SPINOSA | 7 |
| SHELL POWDER | |
| KAOLIN | 25 |
| TALC | 68 |

### Example 8: Softening Oil Booster

TABLE 8
SOFTENING OIL BOOSTER

| INGREDIENT INCI | EX 8: SOFTENING OIL BOOSTER |
|---|---|
| PLANT OR FRUITOIL: | <2.0 |
| CARYOCAR | |
| BRASILIENSE FRUIT | |
| OIL AND/OR | |
| SIMMONDSIA | |
| CHINENSIS (JOJOBA) | |
| SEED OIL | |
| ESTER: ISOPROPYL | 98 |
| MYRISTATE AND/OR | |
| ISOPROPYL | |
| PALMITATE | |
| TOCOPHEROL | 0.0005 |

### Example 9: Acid Base (Paraben Free)

TABLE 9
Acid Base

| INGREDIENT INCI | EX 9: Acid BASE |
|---|---|
| SALICYLIC ACID | 0.2 |
| WATER | Q.S.100 |
| ADDITIVES: | <1.0 |
| FRUIT EXTRACT, | |
| PRESERVATIVES AND | |
| PH ADJUSTER: | |
| POTASSIUM | |
| SORBATE AND/OR | |
| SODIUM BENZOATE AND/OR | |
| CITRIC ACID | |
| VINEGAR | 4 |

### Example 10: Fragrance Booster (Paraben Free)

TABLE 10
FRAGRANCE

| INGREDIENT INCI | EX 10: FRAGRANCE BOOSTER |
|---|---|
| FRAGRANCE | 5 |
| EMOLLIENT: | 95 |
| ISOPROPYL | |
| PALMITATE AND/OR | |
| ISOPROPYL | |
| MYRISTATE | |

### Example 11: Fragrance-Heart (Paraben Free)

TABLE 11
FRAGRANCE-HEART

| INGREDIENT INCI | EX 11: FRAGRANCE HEART |
|---|---|
| FRAGRANCE | 5 |
| EMOLLIENT: | 95 |
| ISOPROPYL | |
| PALMITATE AND/OR | |
| ISOPROPYL | |
| MYRISTATE | |

### Example 12: Fragrance-Fresh (Paraben Free)

TABLE 12
FRAGRANCE-FRESH

| INGREDIENT INCI | EX 12: FRAGRANCE FRESH |
|---|---|
| FRAGRANCE | 5 |
| EMOLLIENT: | 95 |
| ISOPROPYL | |
| PALMITATE AND/OR | |
| ISOPROPYL | |
| MYRISTATE | |

### Example 13: Cleanser #1: Cleanser #1 (Clarifying and Plumping—Right Side)—*Recipe for A Full Head

TABLE 13
Cleanser #1 Recipe for a Full Head

| | Percentage of Formulation |
|---|---|
| Ingredient | (by weight of the formulation) |
| Cleansing Liquid Base | 66.7% |
| Volumizing Starch Booster | 20.0% |
| Plumping Jelly Booster | 13.3% |

### Example 14: Cleanser #2: Cleanser at Roots (Gentle Oil Cleanser—Right Side) *Recipe for a Full Head at Roots Only

TABLE 14
Cleanser #2 Cleanser at Roots

| | Percentage of Formulation |
|---|---|
| Ingredient | (by weight of the formulation) |
| Cleansing Liquid Base | 81.6% |
| Repairing/softening Butter | 16.3% |
| Softening Oil | 2.0% |

### Example 15: Cleanser #3—Cleanser on Ends (Smoothing Defining Cleanser) *Recipe for A Full Head at Ends Only

TABLE 15
Cleanser #3

| | Percentage of Formulation |
|---|---|
| Ingredient | (by weight of the formulation) |
| Cleansing Liquid Base | 76.9% |
| Smoothing Syrup | 23.1% |

*Application: Applied to Dry Hair, Tiny Bit of Water Added to Lather in Sections, Then Rinsed. Followed by Conditioning Milk Alone, Left On 5 Minutes

### Example 16: Cleanser #4—Cleanser (Exfoliating for Dry Hair Focus on Scalp)—*Recipe for a Full Head

TABLE 16
Cleanser #4

| | Percentage of Formulation |
|---|---|
| | (by weight of the |
| Ingredient | formulation) |
| Cleansing Liquid Base | 78.7% |
| Exfoliating Grains | 18.1% |
| Softening Oil | 3.0% |

### Example 17: Cleanser #5: Cleanser (Bodifying)—*Recipe for A Full Head

TABLE 17
Cleanser #5

| | Percentage of |
|---|---|
| | Formulation (by weight |
| Ingredient | of the formulation) |
| Cleansing Liquid Base | 77.8% |
| Volumizing Starch | 14.8% |
| Plumping Jelly | 7.4% |

### Example 18

TABLE 18
Cleanser #6

| | Percentage of |
|---|---|
| | Formulation (by weight |
| Ingredient | of the formulation) |
| Cleansing Liquid Base | 68.9% |
| Volumizing Starch | 17.2% |
| Plumping Jelly | 13.7% |

### Example 19: Cleanser #7: Cleanser (Smoothing and Softening for Thin Hair—Left Side) Recipe for a Full Head

TABLE 19
Cleanser #7

| | Percentage of |
|---|---|
| | Formulation (by weight |
| Ingredient | of the formulation) |
| Cleansing Liquid Base | 84.6% |
| Smoothing Syrup | 14.9% |
| Softening Oil | 0.5% |

### Example 20: Cleanser #8 Smoothing and Softening for Thick Hair

TABLE 20
Cleanser #8

| | Percentage of |
|---|---|
| | Formulation (by |
| | weight of the |
| Ingredient | formulation) |
| Cleansing Liquid Base | 80.4% |
| Smoothing Syrup | 8.3% |
| Softening Oil | 0.14% |
| Volumizing Starch | 11.0% |

*Followed by Conditioning Milk with Just 2 Drops of Softening Oil

### Example 21: Cleanser #9

TABLE 21
Cleanser #9

| | Percentage of |
|---|---|
| | Formulation (by weight |
| Ingredient | of the formulation) |
| Cleansing Liquid Base | 83.9% |
| Smoothing Syrup | 15.5% |
| Softening Oil | 00.5% |

### Example 22: Conditioner #1: Conditioner (Moisturizing, Softening, And Shape Enhancing—All Over)—Recipe for A Full Head

TABLE 22
Conditioner #1

| | Percentage of |
|---|---|
| | Formulation (by weight |
| Ingredient | of the formulation) |
| Conditioning Milk | 88.8% |
| Repairing/softening | 4.4% |
| Butter | |
| Plumping Jelly | 6.6% |

### Example 23: Conditioner #2: Conditioner (Smoothing)—Recipe for A Full Head

TABLE 23
Conditioner #2

| | Percentage of |
|---|---|
| | Formulation (by weight |
| Ingredient | of the formulation) |
| Conditioning Milk | 75.5% |
| Smoothing Syrup | 24.4% |

### Example 24: Conditioner #3: Conditioning (Bodifying)—Right Side—Recipe for A Full Head

TABLE 24
Conditioner #3

| | Percentage of Formulation |
|---|---|
| | (by weight of the |
| Ingredient | formulation) |
| Conditioning Milk | 82.2% |
| Volum izing Starch | 17.7% |

### Example 25: Conditioner #4: Conditioning (Smoothing and Softening—Global) Recipe for A Full Head

TABLE 25
Conditioner #4

| | Percentage of Formulation |
|---|---|
| | (by weight of the |
| Ingredient | formulation) |
| Conditioning Milk | 67.0% |
| Smoothing Syrup | 12.2% |
| Plumping Jelly | 15.0% |
| Repairing/softening | 5.5% |
| Butter | |

### Example 26: Conditioner #5: Smoothing for Coarse Hair on the Root Area that was not Relaxed)—*Recipe for a Full Head at the Roots

TABLE 26
Conditioner #5

| | Percentage of Formulation |
|---|---|
| | (by weight of the |
| Ingredient | formulation) |
| Conditioning Milk | 82.2% |
| Repairing/softening | 17.7% |
| Butter | |

### Example 27: Conditioner #6: Smoothing for Coarse Hair on the Relaxed Area) Recipe for a Full Head

TABLE 27
Conditioner #6

| | Percentage of Formulation |
|---|---|
| Ingredient | (by weight of the formulation) |
| Conditioning Milk | 78.7% |
| Smoothing Syrup | 21.2% |

### Example 28

TABLE 28
Conditioner #7

| | Percentage of Formulation |
|---|---|
| Ingredient | (by weight of the formulation) |
| Conditioning Milk | 99.7% |
| Softening Oil | 0.3% |

### Example 29

TABLE 29
CONVERSIONS FROM METRIC TO DISPENSER UNITS

| | | METRIC | | DISPENSER |
|---|---|---|---|---|
| COMPONENT | INGREDIENT | AMOUNT | | UNIT |
| CLEANSING BASE | CLEANSING LIQUID | 18 | ml | 1 tbsp + 1 tsp |
| CONDITIONING MILK | CONDITIONING MILK | 18.5 | ml | 1 tbsp + 1 tsp |
| VOLUMIZING BOOSTER | VOLUMIZING STARCH | 5 | g | 2 tbsp |
| EXFOLIATING BOOSTER | EXFOLIATING GRAINS | 3 | g | 1 1/2 tbsp |
| PLUMPING BOOSTER | PLUMPING JELLY | 2 | ml | 1/4 tsp |
| SMOOTHING BOOSTER | SMOOTHING SYRUP | 2 | ml | 1/4 tsp |
| SOFTENING BOOSTER | SOFTENING OIL | 0.025 | g | 1 drop |
| REPAIRING/SOFTENING BOOSTER | REPAIRING/SOFTENING BUTTER | 4 | ml | 1 tsp |

### Example 30: Hair Care Bundle 1

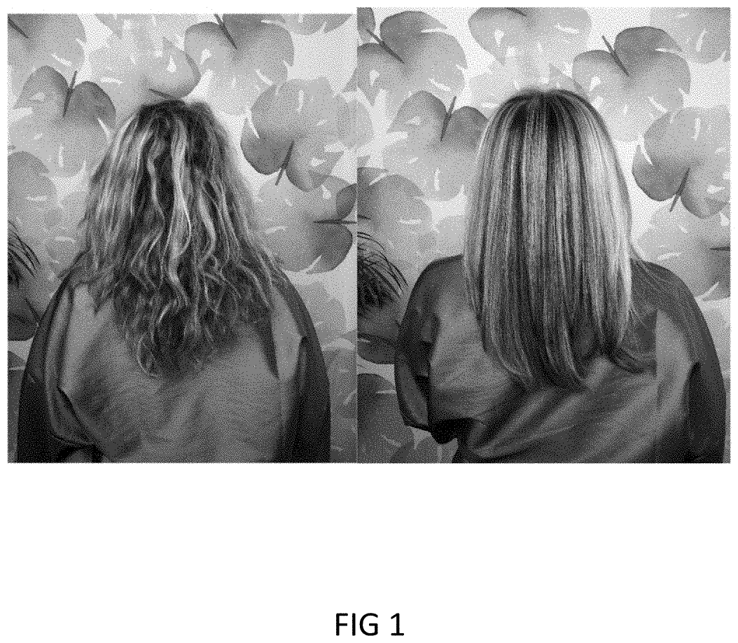

Referring now to FIG. 1, as a control the subject's hair was first cleansed, conditioned and styled according to their usual care routine; in the salon setting, the subject received care using the below listed bundle, followed by drying and styling.

TABLE 30
Bundle #1
EX 30: HAIR CARE BUNDLE #1

Cleanser #1
Conditioner #1

### Example 31: Hair Care Bundle #2

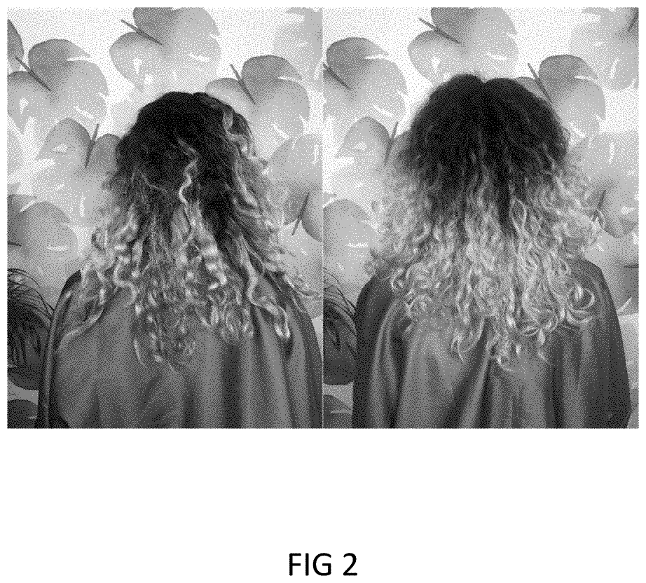

Referring now to FIG. 2, as a control the subject's hair was first cleansed, conditioned and styled according to their usual care routine; in the salon setting, the subject received care using the below listed bundle, followed by drying and styling.

TABLE 31
Bundle #2
EX 31: HAIR CARE BUNDLE #2
Cleanser #2 At Roots only:
Cleanser #3 At Ends Only:
Conditioning Milk Alone, Left On at least 5
Minutes

### Example 32: Hair Care Bundle #3

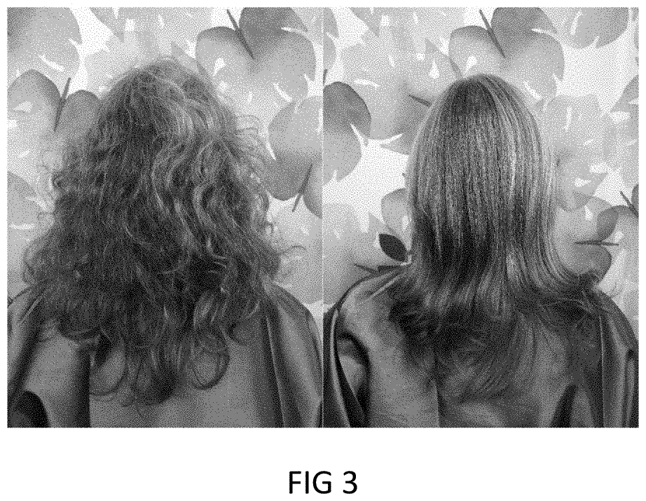

Referring now to FIG. 3, as a control the subject's hair was first cleansed, conditioned and styled according to their usual care routine (left image); in the salon setting, the subject received care using the below listed bundle (right image), followed by drying and styling.

TABLE 32
Bundle #3
EX 32: HAIR CARE BUNDLE #3
Cleanser #4
Conditioner #2

### Example 33: Hair Care Bundle

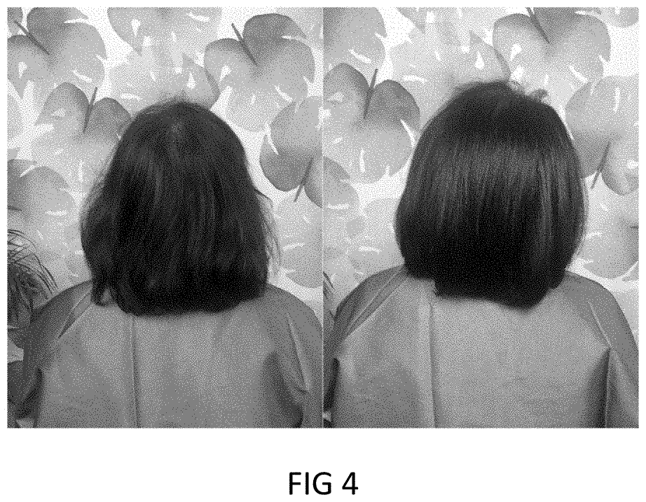

Referring now to FIG. 4, as a control the subject's hair was first cleansed, conditioned and styled according to their usual care routine (left image); in the salon setting, the subject received care using the below listed bundle (right image), followed by drying and styling.

TABLE 33
Bundle #4
EX 33: HAIR CARE BUNDLE #4
Cleanser #5
Conditioner #3

### Example 34: Hair Care Bundle

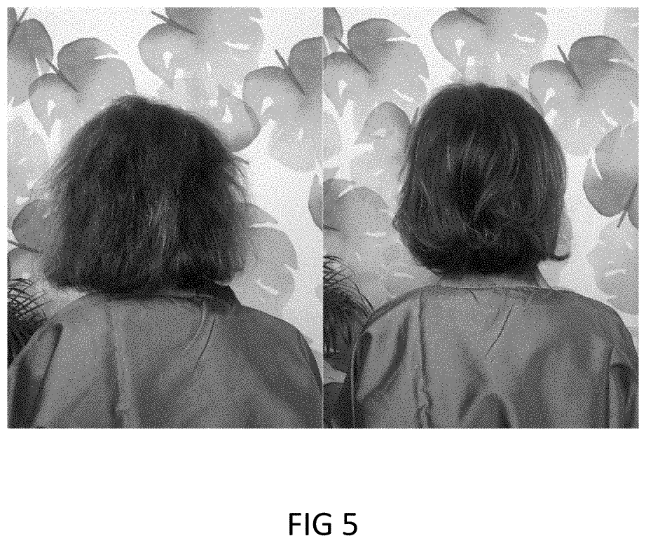

Referring now to FIG. 5, as a control the subject's hair was first cleansed, conditioned and styled according to their usual care routine (left image); in the salon setting, the subject received care using the below listed bundle (right image), followed by drying and styling.

TABLE 34
Bundle #5
EX 34: HAIR CARE BUNDLE #5
Cleanser #5

### Example 35: Hair Care Bundle 6

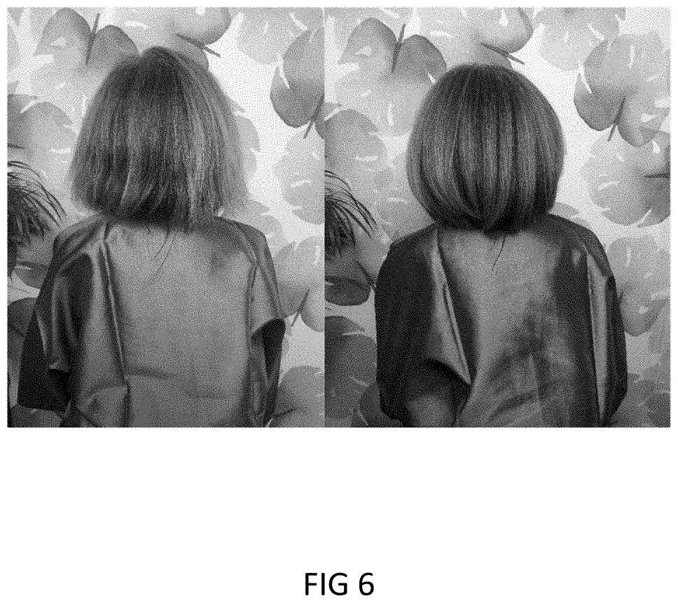

Referring now to FIG. 6, as a control the subject's hair was first cleansed, conditioned and styled according to their usual care routine (left image); in the salon setting, the subject received care using the below listed bundle (right image), followed by drying and styling.

TABLE 35
Bundle #6
EX 35: HARE CARE BUNDLE #6
Cleanser #7
Conditioner #4

### Example 36: Hair Care Bundle 7

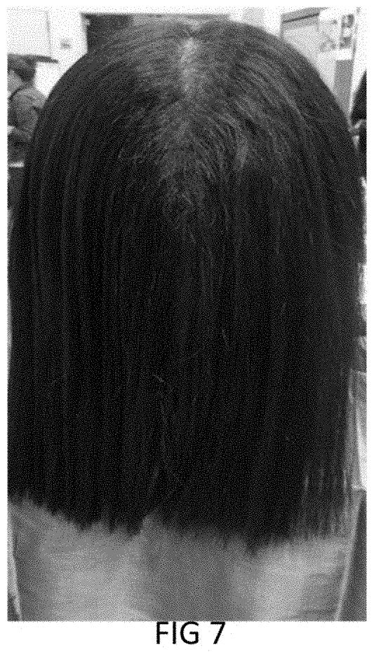

Referring now to FIG. 7, as a control the subject's hair was first cleansed, conditioned and styled according to their usual care routine (left image); in the salon setting, the subject received care using the below listed bundle (right image), followed by drying and styling.

TABLE 36
Bundle #7
EX 36: HARE CARE BUNDLE #7
Conditioner #5
Conditioner #6

### Example 37: Hair Care Bundle 8

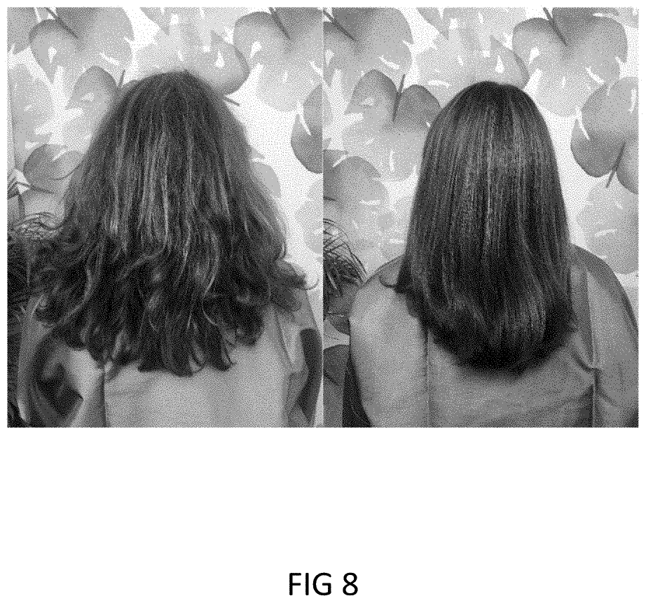

Referring now to FIG. 8, in the salon setting, the subject received care using the below listed bundle, followed by drying and styling.

TABLE 37
Bundle #8
EX 37: HARE CARE BUNDLE #8
Cleanser #8
Conditioner #7
Acid Base (Example #9)

### Example 38: Hair Care Bundle 9

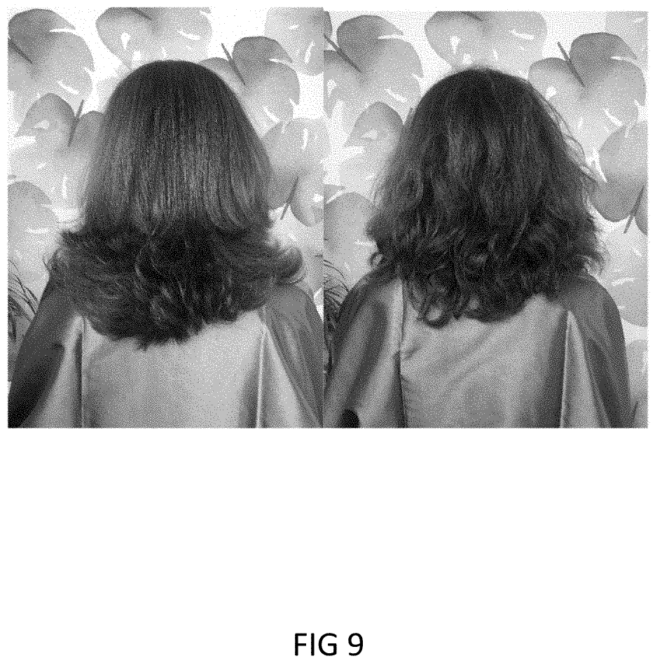

Referring now to FIG. 9, as a control the subject's hair was first cleansed, conditioned and styled according to their usual care routine (left image); in the salon setting, the subject received care using the below listed bundle (right image), followed by drying and styling.

TABLE 38
Bundle #9
EX 38: HARE CARE BUNDLE #9
Cleanser #9

### Example 39: Wet Combing Forces

Smoothness properties in the wet state were evaluated for hair tresses treated with treatments below to be compared with control shampoo alone, via wet combing (Garcia Method).

Treatment steps included:
1. Eight (8) European medium brown hair tresses (3.0 g, 8″ length, 1″ wide) per each of treatment Groups 1, 2 and 3. A 6% bleach was applied on each tress prior to treatment. Referring to TABLE 39B, each of 8 tresses for each treatment Group was treated as indicated.
2. After treatment, tresses were tested by Wet combing. The primary technical function of most conditioning products is to lubricate the hair surface; and, in doing so, facilitate manageability and provide detangling benefits and lower combing friction. A common and highly consumer-relevant approach for measuring this lubrication involves an instrumental combing experiment.
3. Combing experiments were performed on each swatch in the wet state after treatment.
4. Six (6) measurements were taken per swatch using an Instron tensile tester to measure frictional forces while a hair tress is pulled through a comb in accordance with the widely-used method first proposed by Garcia & Diaz (JSCC, 27, (1976) 379-398—Combability Measurements on Hair).
5. Measurement data were analyzed and represented in box & whisker plots (converted to tabular format for presentation herein) that were created using a software statistical analysis was performed on the data. Statistics were performed using the student's t-test at the 95% confidence level.

In the examples 39-41 herein, the following conventional shampoo formula is used as a control, as indicated (DOP™):

TABLE 39A

| INCI US | Shampoo |
|---|---|
| LAURETH-12 | 0.3 |
| COCAMIDE MEA | 0.5 |
| SODIUM LAURETH SULFATE | 8.4 |
| HEXYLENE GLYCOL | 1.3 |
| COCO-BETAINE | 3.0 |
| GLYCERIN | 1.0 |
| WATER | QS 100 |
| ADDITIVES: SALICYLIC ACID, | <5.0 |
| FRAGRANCE, PRESERVATIVES, | |
| VITAMINS, PH ADJUSTER, | |
| NEUTRALIZERS, COLORANTS, | |
| SODIUM CHLORIDE | |
| CITRIC ACID | 0 (Adjuster) |

TABLE 39B
Test Conditions

| Treatment | Formulation | Amt | Process 1 |
|---|---|---|---|
| CONTROL (w | | | |
| Conditioner) | | | |
| Shampoo | Cleansing | 0.25 ml/g of hair | 30 sec massage; |
| | | | 30 sec rinse |
| Conditioner | Conditioning | 0.25 ml/g of hair | 30 sec massage; |
| | or Treatment | | 30 sec rinse |
| | Milk | | |
| INVENTIVE | | | |
| TREATMENT | | | |
| (with | | | |
| conditioner | | | |
| plus additive) | | | |
| Additive | Smoothing | 1 tsp in 20 ml in each | |
| | Syrup | of shampoo and | |
| | | conditioner | |
| Shampoo | Cleansing | 0.32 ml/g of hair | 30 sec massage; |
| | | | 30 sec rinse |
| Conditioner | Conditioning | 0.32 ml/g of hair | 30 sec massage; |
| | or Treatment | | 30 sec rinse |
| | Milk | | |
| Control (no | | | |
| conditioner) | | | |
| Shampoo | DOP^{TM} | 0.4 ml/g of hair | 30 sec massage, |
| | Shampoo | | 1 min leave-in’ |
| | | | 30 sec rinse |

TABLE 39C
Results

| | | Maximum | | |
|---|---|---|---|---|
| | | Load | | Std Error |
| Treatment | Formulation | (mean) | Std Dev | Mean |
| CONTROL (w | | 19.73 | 0.6 | 0.21 |
| Conditioner) | | | | |
| Shampoo | Cleansing | | | |
| Conditioner | Conditioning | | | |
| | or Treatment | | | |
| | Milk | | | |
| INVENTIVE | | 15.16 | 0.33 | 0.12 |
| TREATMENT | | | | |
| (with | | | | |
| conditioner | | | | |
| plus additive) | | | | |
| Additive | Smoothing | | | |
| | Syrup | | | |
| Shampoo | Cleansing | | | |
| Conditioner | Conditioning | | | |
| | or Treatment | | | |
| | Milk | | | |
| Control (no | | 124.30 | | |
| conditioner) | | | | |
| Shampoo | DOP^{TM} | | | |
| | Shampoo | | | |

Results: Referring now to TABLE 39C, the data shows the maximum load (mean) of combing forces (gf) measured for control and inventive examples. There was a statistically significant difference between the control and the inventive treatment groups. The represented values are based on the average mean for each treatment.

Percent reduction in combing force was calculated, where Percent (%) reduction in combing force=(1−combing force of treatment/combing force of control)×100.

A decrease in wet combing force of approximately 23% was measured for the inventive treatment group relative to the control. In accordance with the various embodiments, use of a smoothing syrup together with one or both micellar cleansing and treatment milk compositions according to the disclosure provides a decrease in dry combing force of about 18% to about 28%.

### Example 40: Dry Combing Forces

Smoothness properties in the dry state were evaluated for hair tresses treated with treatments below to be compared with control shampoo alone, via Dry combing.

Treatment steps included:
1. Eight (8) European medium brown hair tresses (3.0 g, 8″ length, 1″ wide) per each of treatment Groups 1, 2 and 3. A 6% bleach was on each tress prior to treatment. Referring to TABLE 40A, each of 8 tresses for each treatment Group were treated as indicated. The control composition is shown in TABLE 39A.
2. After treatment with control and inventive compositions as shown in TABLE 40A, tresses were air dried overnight at 60% relative humidity.
3. Combing experiments were performed on each swatch in the dry state after overnight drying.
4. Six (6) measurements were taken per swatch using an Instron tensile tester to evaluate performance according to the Garcia method, as described above.
5. Measurement data were analyzed and represented in box & whisker plots (converted to tabular format for presentation herein) that were created using a software statistical analysis was performed on the data. Statistics were performed using the student's t-test at the 95% confidence level.

TABLE 40A
Test Conditions

| Treatment | Formulation | Amt | Process 1 |
|---|---|---|---|
| CONTROL (w | | | |
| Conditioner) | | | |
| Shampoo | Cleansing | 0.25 ml/g of hair | 30 sec massage; |
| | | | 30 sec rinse |
| Conditioner | Conditioning | 0.25 ml/g of hair | 30 sec massage; |
| | or Treatment | | 30 sec rinse |
| | Milk | | |
| INVENTIVE | | | |
| TREATMENT | | | |
| (with | | | |
| conditioner | | | |
| plus additive) | | | |
| Additive | Softening Oil | 5 drops in 20 ml in | |
| | | each of shampoo | |
| | | and conditioner | |
| Shampoo | Cleansing | 0.27 ml/g of hair | 30 sec massage; |
| | | | 30 sec rinse |
| Conditioner | Conditioning | 0.27 ml/g of hair | 30 sec massage; |
| | or | | 30 sec rinse |
| | Treatment Milk | | |
| Control (no | | | |
| conditioner) | | | |
| Shampoo | DOP^{TM} | 0.4 ml/g of hair | 30 sec massage, |
| | Shampoo | | 1 min leave-in’ |
| | | | 30 sec rinse |

TABLE 40B
Results

| | | Maximum | | |
|---|---|---|---|---|
| | | Load | | Std Error |
| Treatment | Formulation | (mean) | Std Dev | Mean |
| CONTROL (w | | 48.2 | 2.99 | 1.06 |
| Conditioner) | | | | |
| Shampoo | Cleansing | | | |
| Conditioner | Conditioning | | | |
| | or | | | |
| | Treatment Milk | | | |
| INVENTIVE | | 41.02 | 2.72 | 0.96 |
| TREATMENT | | | | |
| (with | | | | |
| conditioner | | | | |
| plus additive) | | | | |
| Additive | Softening Oil | | | |
| Shampoo | Cleansing | | | |
| Conditioner | Conditioning | | | |
| | or | | | |
| | Treatment Milk | | | |
| Control (no | | | | |
| conditioner) | | | | |
| Shampoo | DOP^{TM} | | | |
| Shampoo | | | | |

Results: Referring now to TABLE 40B, the data shows the maximum load (mean) of combing forces (gf) measured for control and inventive examples. There was a statistically significant difference between the control and the inventive treatment groups. The represented values are based on the average mean for each treatment.

Percent reduction in combing force was calculated, where Percent (%) reduction in combing force=(1−combing force of treatment/combing force of control)×100.

A decrease in dry combing force of approximately 15% was measured for the inventive treatment group relative to the control. In accordance with the various embodiments, use of a softening oil together with one or both micellar cleansing and treatment milk compositions according to the disclosure provides a decrease in dry combing force of about 10% to about 20%.

### Example 41: Dry Repeated Grooming

Anti-breakage properties in the dry state were evaluated for hair tresses treated using a dry grooming [repeated grooming] method.

Treatment steps included:
1. Ten (10) European medium brown hair tresses (3.0 g, 8″ length, 1″ wide) per each of treatment Groups 1, 2 and 3. Referring to TABLE 41A, each of 8 tresses for each treatment Group were treated as indicated. The control composition is shown in TABLE 39A.
2. After treatment with control and inventive compositions as shown in TABLE 41A, tresses were air dried overnight at 60% relative humidity.
3. Repeated grooming was employed using an automatic comber for a total of 10,000 cycles.
4. Fibers were collected and assessed after every 1,000 cycles. The number of broken fibers is recorded as a function of repeated combing/brushing strokes. Any treatment that reduces snags, entanglements and abrasion can help in substantially lowering the number of broken fibers.
5. Measurement data were analyzed and represented in box & whisker plots (converted to tabular format for presentation herein) that were created using a software statistical analysis was performed on the data. Statistics were performed using the student's t-test at the 95% confidence level.

TABLE 41A
Test Conditions

| Treatment | Formulation | Amt | Process 1 |
|---|---|---|---|
| CONTROL (w | | | |
| Conditioner) | | | |
| Shampoo | Cleansing | 0.25 ml/g of hair | 30 sec massage; |
| | | | 30 sec rinse |
| Conditioner | Conditioning | 0.25 ml/g of hair | 30 sec massage; |
| | or | | 30 sec rinse |
| | Treatment Milk | | |
| INVENTIVE | | | |
| TREATMENT | | | |
| (with | | | |
| conditioner | | | |
| plus additive) | | | |
| Additive | Repairing | 1/4 tsp in 20 ml in | |
| | Butter | each of shampoo | |
| | | and conditioner | |
| Shampoo | Cleansing | 0.27m1/g of hair | 30 sec massage; |
| | | | 30 sec rinse |
| Conditioner | Conditioning | 0.27m1/g of hair | 30 sec massage; |
| | or | | 30 sec rinse |
| | Treatment Milk | | |
| Control (no | | | |
| conditioner) | | | |
| Shampoo | DOP^{TM} | 0.4 ml/g of hair | 30 sec massage, |
| | Shampoo | | 1 min leave-in’ |
| | | | 30 sec rinse |

TABLE 41B
Results

| | | # Broken | | Std Error |
|---|---|---|---|---|
| Treatment | Formulation | Fibers* | Std Dev | Mean |
| CONTROL (w | | 99 | 15 | 5 |
| Conditioner) | | | | |
| Shampoo | Cleansing | | | |
| Conditioner | Conditioning | | | |
| | or | | | |
| | Treatment Milk | | | |
| INVENTIVE | | 50 | 10 | 3 |
| TREATMENT | | | | |
| (with | | | | |
| conditioner | | | | |
| plus additive) | | | | |
| Additive | Repairing | | | |
| | Butter | | | |
| Shampoo | Cleansing | | | |
| Conditioner | Conditioning | | | |
| | or | | | |
| | Treatment Milk | | | |
| Control (no | | 104 | | |
| conditioner) | | | | |
| Shampoo | DOP^{TM} | | | |
| | Shampoo | | | |

*after 10,000 brush strokes

Results: Referring now to TABLE 41B, the table shows the number of fibers broken after a total of 10,000 brush strokes (mean) for control and inventive examples. There was a statistically significant difference between the control and the inventive treatment groups. The represented values are based on the average mean for each treatment.

Percent reduction in breakage was calculated, where Percent (%) reduction in breakage=(1−mean % of broken fibers of treatment/mean % of broken fibers of control)×100.

A decrease in breakage of approximately 49% was measured for the inventive treatment group relative to the control. In accordance with the various embodiments, use of a repairing butter together with one or both micellar cleansing and treatment milk compositions according to the disclosure provides a decrease in breakage of about 35% to about 55%.

### Example 42: Smoothing Recipe Before and after

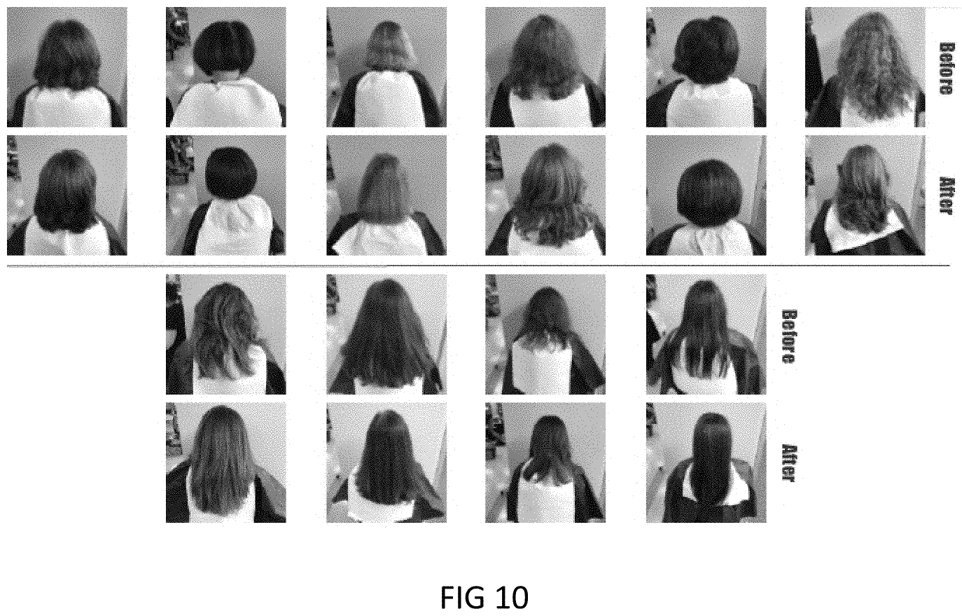

Referring now to FIG. 10, a series of “before” and “after” photographic images are provided for 10 test subjects. As a control for each subject, each subject's hair was first cleansed, conditioned and styled according to their usual care routine (top image); in the salon setting, the subject received care using the below listed bundle (test inventive composition; bottom image), followed by drying and styling.

Smoothing Recipe

Treatment Milk

Smoothing Syrup

Plumping Jelly

Repairing Butter

Fragrance

Evident improvement in smoothing of hair can be seen for each subject.

### Example 43: Repair Recipe Before and after

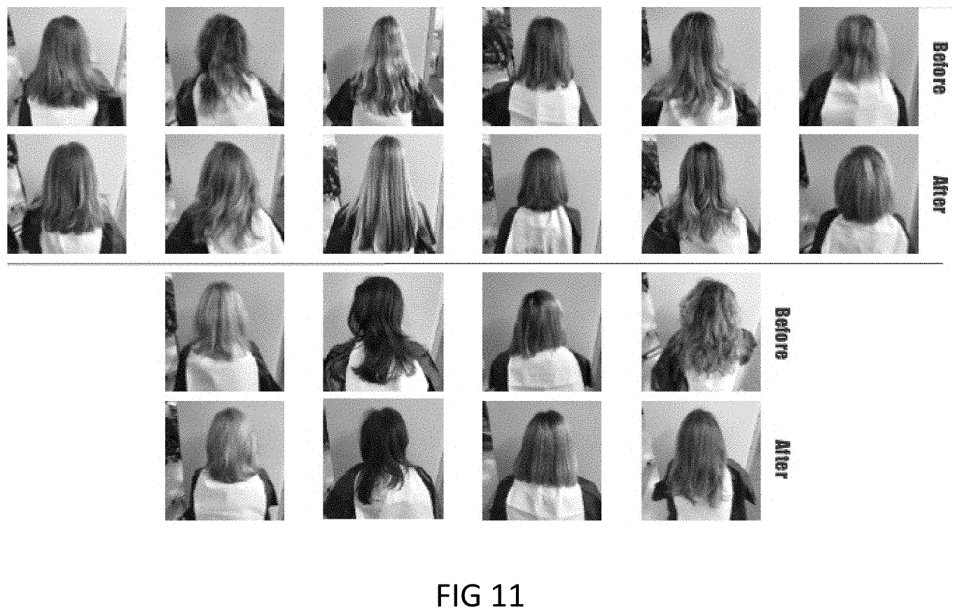

Referring now to FIG. 11, a series of “before” and “after” photographic images are provided for 10 test subjects. As a control for each subject, each subject's hair was first cleansed, conditioned and styled according to their usual care routine (top image); in the salon setting, the subject received care using the below listed bundle (test inventive composition; bottom image), followed by drying and styling.

Repair Recipe

Treatment Milk

Plumping Jelly

Repairing Butter

Fragrance

Evident improvement in smoothing of hair can be seen for each subject.

### Example 44: Volume Recipe Before and after

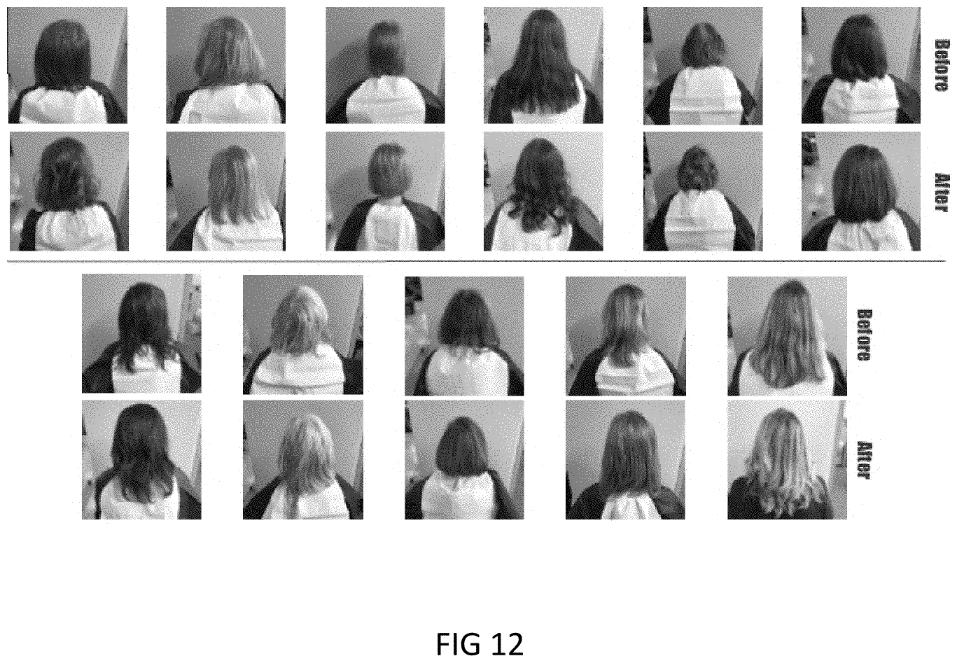

Referring now to FIG. 12, a series of “before” and “after” photographic images are provided for 11 test subjects. As a control for each subject, each subject's hair was first cleansed, conditioned and styled according to their usual care routine (top image); in the salon setting, the subject received care using the below listed bundle (test inventive composition; bottom image), followed by drying and styling.

Volume Recipe

Cleansing Liquid

Exfoliating Grains

Softening Oil

Fragrance

Evident improvement in smoothing of hair can be seen for each subject.

While the disclosure has been described with reference to a preferred embodiment, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9% 11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1) A system for formulating a hair care product, comprising:
a) a hair product formulary schedule comprising a plurality of hair care formulation recipes, each hair care formulation recipe including instructions for combining one of a plurality of aqueous base compositions, and one or a combination of water dispersible cosmeticity booster compositions;
b) a plurality of aqueous base compositions comprising at least one cleansing base and at least one conditioning base, each aqueous base composition disposed in a separate dispensing container; and
c) a plurality of water dispersible cosmeticity booster compositions, each being readily dispersible into the base compositions and each disposed in a separate dispensing container, the plurality of water dispersible cosmeticity booster compositions comprising active ingredients corresponding to hair target attributes.

2) The system for formulating a hair care product according to claim 1, comprising one or more additional base compositions.

3) The system for formulating a hair care product according to claim 1, wherein:
a) the plurality of aqueous base compositions comprises one or more of:
i) a cleansing base composition that comprises at least one alkyl polyglucoside, and at least one cationic polymer, water and optionally at least one active; and
ii) a conditioning base composition that comprises at least one cationic surfactant including cationizable surfactants together with an acid neutralizer, at least one fatty alcohol, and water, and optionally at least one active
wherein all percentages are by weight based upon the weight of the booster composition.

4) The system for formulating a hair care product according to claim 1, wherein:
a) the plurality of aqueous base compositions comprises one or more of:
i) a cleansing base composition that comprises at least one alkyl polyglucoside surfactant, and a gum derivative; and
ii) a conditioning base composition that comprises at least one fatty alcohol, at least one humectant, and at least one organic acid;
wherein all percentages are by weight based upon the weight of the booster composition.

5) The system for formulating a hair care product according to claim 1, the system comprising a plurality of water dispersible cosmeticity booster compositions that correspond, respectively, to hair target attributes that include exfoliating, volumizing, plumping, smoothing, softening, repairing/softening, caring, manageability, and scenting.

6) The system for formulating a hair care product according to claim 5, wherein:
a) the plurality of water dispersible cosmeticity booster compositions comprises two or more of:
i) the booster composition corresponding to exfoliating that includes a powder, at least one clay, at least one filler, and starch; and
ii) the booster composition corresponding to volumizing that includes sodium bicarbonate, an organic acid, and starch; and
iii) the booster composition corresponding to plumping that includes at least one polymeric thickener, in some embodiments comprising a polysaccharide, in some embodiments comprising a polysaccharide gellifying thickener, together with aloe, one or a combination of polyols, and optionally an active comprising a beta hydroxy acid; and
iv) the booster composition corresponding to smoothing that includes at least one gum, and at least one hydroxy group containing compound selected from polyols, longer chain monoalcohols (C₈ to C₃₀) which include fatty alcohols, and combinations thereof; and
v) the booster composition corresponding to repairing butter that includes an anhydrous blend of plant/vegetable oils, and optionally at least one active comprising a vitamin; and
vi) the booster composition corresponding to repairing/softening oil that includes an anhydrous blend comprising at least one plant oil, at least one polar emollient ester, and optionally at least one active comprising a vitamin, comprising tocopherol; and
vii) the booster composition corresponding to scenting that includes an essentially anhydrous blend of a fragrance with at least one polar emollient selected from isopropyl palmitate, isopropyl myristate, and combinations thereof.

7) The system for formulating a hair care product according to claim 5, wherein:
a) the plurality of water dispersible cosmeticity booster compositions comprises one or more of:
i) the booster composition corresponding to exfoliating that comprises at least one or a combination of cosmetic powders and clays; and
ii) the booster composition corresponding to volumizing that comprises at least one starch; and
iii) the booster composition corresponding to plumping that comprises aloe, and at least one humectant; and
iv) the booster composition corresponding to smoothing that comprises at least one gum, at least one fatty alcohol emollient and at least one humectant; and
v) the booster composition corresponding to softening that comprises at least one polar emollient ester and at least one plant or fruit oil; and
vi) the booster composition corresponding to repairing/softening that comprises a combination of plant or fruit oils comprising one or more of avocado oil and coconut oil; and
vii) the booster composition corresponding to scenting that includes an essentially anhydrous blend of a fragrance with at least one polar emollient.

8) A system for formulating a hair cleansing product, comprising:
a) a hair product formulary schedule comprising a plurality of hair care formulation recipes, each hair care formulation recipe including instructions for combining an aqueous cleansing base composition, and one or a combination of water dispersible cosmeticity booster compositions;
b) an aqueous cleansing base composition disposed in a dispensing container, the dispensing container adapted to dispense a fixed unit of cleansing base composition, the aqueous base cleansing composition comprising: at least one alkyl polyglucoside, at least one cationic polymer, water and optionally at least one active; and
c) a plurality of water dispersible cosmeticity booster compositions, each being readily dispersible into the base composition and each disposed in a separate dispensing container, each dispensing container that is adapted to dispense a fixed unit of the booster composition, the plurality of water dispersible cosmeticity booster compositions comprising active ingredients corresponding to hair target attributes that include exfoliating, volumizing, plumping, smoothing, softening, repairing/softening, caring, manageability, and scenting, the plurality of water dispersible cosmeticity booster compositions comprising two or more of:
i) the booster composition corresponding to exfoliating that includes a powder, at least one clay, at least one filler, and starch; and
ii) the booster composition corresponding to volumizing that includes sodium bicarbonate, an organic acid, and starch; and
iii) the booster composition corresponding to plumping that includes at least one polymeric thickener, in some embodiments comprising a polysaccharide, in some embodiments comprising a polysaccharide gellifying thickener, together with aloe, one or a combination of polyols, and optionally an active comprising an organic acid; and
iv) the booster composition corresponding to smoothing that includes at least one gum, and at least one hydroxy group containing compound selected from polyols, longer chain monoalcohols (C₈ to C₃₀) which include fatty alcohols, and combinations thereof; and
v) the booster composition corresponding to repairing butter that includes an anhydrous blend of plant/vegetable oils, and optionally at least one active comprising a vitamin; and
vi) the booster composition corresponding to repairing/softening oil that includes an anhydrous blend comprising at least one plant or fruit oil, at least one polar emollient ester, and optionally at least one active comprising a vitamin, comprising tocopherol; and
vii) the booster composition corresponding to scenting that includes an essentially anhydrous or anhydrous blend of a fragrance with at least one polar emollient selected from isopropyl palmitate, isopropyl myristate, and combinations thereof.

9) The system for formulating a hair cleansing product according to claim 8, wherein the cleansing base composition comprises
i) at least one alkyl polyglucoside surfactant,
ii) organic acid, and
iii) at least one gum derivative; and
wherein all percentages are by weight based upon the weight of the conditioning base composition, and
wherein one or more of:
i) the booster composition corresponding to exfoliating that comprises at least one or a combination of cosmetic powders and clays; and
ii) the booster composition corresponding to volumizing that comprises at least one starch; and
iii) the booster composition corresponding to plumping that comprises aloe, and at least one humectant; and
iv) the booster composition corresponding to smoothing that comprises at least one gum, at least one fatty alcohol emollient and at least one humectant; and
v) the booster composition corresponding to softening that comprises at least one polar emollient ester and at least one plant or fruit oil; and
vi) the booster composition corresponding to repairing/softening that comprises a combination of plant or fruit oils comprising one or more of avocado oil and coconut oil; and
vii) the booster composition corresponding to scenting that includes an essentially anhydrous blend of a fragrance with at least one polar emollient.

10) The system for formulating a hair cleansing product according to claim 8, wherein the hair product formulary schedule comprises a plurality of hair cleansing formulation recipes selected from the group consisting of:
i) a hair cleansing formulation comprising a predetermined number of dispensed units of a paraben-free cleansing base, a predetermined number of dispensed units of a volumizing booster, and a predetermined number of dispensed units of a plumping booster;
ii) a hair cleansing formulation comprising a predetermined number of dispensed units of a paraben-free cleansing base, and a predetermined number of dispensed units of a repairing/softening booster;
iii) a hair cleansing formulation comprising a predetermined number of dispensed units of a paraben-free cleansing base, and a predetermined number of dispensed units of a smoothing booster;
iv) a hair cleansing formulation comprising a predetermined number of dispensed units of a paraben-free cleansing base, and a predetermined number of dispensed units of an exfoliating booster;
v) a hair cleansing formulation comprising a predetermined number of dispensed units of a paraben-free cleansing base, a volumizing booster, and a predetermined number of dispensed units of a plumping booster;
vi) a hair cleansing formulation comprising a predetermined number of dispensed units of a paraben-free cleansing base, a predetermined number of dispensed units of a volumizing booster, and a predetermined number of dispensed units of a plumping booster;
vii) a hair cleansing formulation comprising a predetermined number of dispensed units of a paraben-free cleansing base, a predetermined number of dispensed units of a smoothing booster and a predetermined number of dispensed units of a softening booster;
viii) a hair cleansing formulation comprising a predetermined number of dispensed units of a paraben-free cleansing base, a predetermined number of dispensed units of a smoothing booster, a predetermined number of dispensed units of a softening booster, and a predetermined number of dispensed units of a volumizing booster; and
ix) a hair cleansing formulation comprising a predetermined number of dispensed units of a paraben-free cleansing base, a predetermined number of dispensed units of a smoothing booster, and a predetermined number of dispensed units of a softening booster.

11) A system for formulating a hair care conditioning product, comprising:
a) a hair product formulary schedule comprising a plurality of hair care formulation recipes, each hair care formulation recipe including instructions for combining an aqueous conditioning base composition and one or a combination of water dispersible cosmeticity booster compositions;
b) an aqueous conditioning base composition disposed in a dispensing container and adapted to dispense a fixed unit of conditioning base composition, the aqueous base conditioning base composition comprising at least one cationic surfactant including cationizable surfactants together with an acid neutralizer, at least one fatty alcohol, and water, and optionally at least one active; and
c) a plurality of water dispersible cosmeticity booster compositions, each being readily dispersible into the base composition and each disposed in a separate dispensing container, the plurality of water dispersible cosmeticity booster compositions comprising active ingredients corresponding to hair target attributes that include exfoliating, volumizing, plumping, smoothing, softening, repairing/softening, caring, manageability, and scenting, the plurality of water dispersible cosmeticity booster compositions comprising two or more of:
i) the booster composition corresponding to exfoliating that includes a powder, at least one clay, at least one filler, and starch; and
ii) the booster composition corresponding to volumizing that includes sodium bicarbonate, an organic acid, and starch; and
iii) the booster composition corresponding to plumping that includes at least one polymeric thickener, in some embodiments comprising a polysaccharide, in some embodiments comprising a polysaccharide gellifying thickener, together with aloe, one or a combination of polyols, and optionally an active comprising an organic acid, and water; and
iv) the booster composition corresponding to smoothing that includes at least one gum, and at least one hydroxy group containing compound selected from polyols, longer chain monoalcohols (C₈ to C₃₀) which include fatty alcohols, and combinations thereof, and water; and
v) the booster composition corresponding to repairing butter that includes an anhydrous blend of plant/vegetable oils, and optionally at least one active comprising a vitamin; and
vi) the booster composition corresponding to repairing/softening oil that includes an anhydrous blend comprising at least one plant oil, at least one polar emollient ester, and optionally at least one active comprising a vitamin, comprising tocopherol; and
vii) the booster composition corresponding to scenting that includes an essentially anhydrous or anhydrous blend of a fragrance with at least one polar emollient selected from isopropyl palmitate, isopropyl myristate, and combinations thereof.

12) The system for formulating a hair care conditioning product according to claim 11, wherein the conditioning base composition comprises
i) at least one fatty alcohol,
ii) at least one humectant, and
iii) at least one organic acid;
wherein all percentages are by weight based upon the weight of the conditioning base composition, and
wherein one or more of:
i) the booster composition corresponding to exfoliating that comprises at least one or a combination of cosmetic powders and clays; and
ii) the booster composition corresponding to volumizing that comprises at least one starch; and
iii) the booster composition corresponding to plumping that comprises aloe, salicylic acid, and at least one humectant; and
iv) the booster composition corresponding to smoothing that comprises at least one gum, at least one fatty alcohol emollient and at least one humectant; and
v) the booster composition corresponding to softening that comprises at least one polar emollient ester and at least one plant or fruit oil; and
vi) the booster composition corresponding to repairing/softening that comprises a combination of plant or fruit oil comprising one or more of avocado oil and coconut oil; and
vii) the booster composition corresponding to scenting that includes an essentially anhydrous blend of a fragrance with at least one polar emollient.

13) The system for formulating a hair care conditioning product according to claim 12, wherein, the hair product formulary schedule comprises a plurality of hair conditioning formulation recipes selected from the group consisting of:
i) a hair conditioning formulation comprising a predetermined number of dispensed units of a paraben-free conditioning base, a predetermined number of dispensed units of a repairing/softening booster, and a predetermined number of dispensed units of a plumping booster;
ii) a hair conditioning formulation comprising a predetermined number of dispensed units of a paraben-free conditioning base and a predetermined number of dispensed units of a smoothing booster;
iii) a hair conditioning formulation comprising a predetermined number of dispensed units of a paraben-free conditioning base and a predetermined number of dispensed units of a volumizing booster;
iv) a hair conditioning formulation comprising a predetermined number of dispensed units of a paraben-free conditioning base, a predetermined number of dispensed units of a smoothing booster, a plumping booster, and a predetermined number of dispensed units of a repairing/softening booster;
v) a hair conditioning formulation comprising a predetermined number of dispensed units of a paraben-free conditioning base and a repairing/softening booster;
vi) a hair conditioning formulation comprising a predetermined number of dispensed units of a paraben-free conditioning base and a smoothing booster.

14) A method for delivering hair care, the method comprising the steps of:
(I) In any order, providing:
a) a hair product formulary schedule comprising a plurality of hair care formulation recipes, each hair care formulation recipe including instructions for combining one of a plurality of aqueous base compositions, and one or a combination of water dispersible cosmeticity booster compositions;
b) an aqueous base composition disposed in a dispensing container, the dispensing container adapted to dispense a fixed unit of aqueous base composition, the aqueous base a cleansing base composition that comprises one of:
i) a cleansing base composition that comprises at least one alkyl polyglucoside, and at least one cationic polymer, water and optionally at least one active; and
ii) a conditioning base composition that comprises at least one cationic surfactant including cationizable surfactants together with an acid neutralizer, at least one fatty alcohol, and water, and optionally at least one active
wherein all percentages are by weight based upon the weight of the aqueous base composition;
c) a plurality of water dispersible cosmeticity booster compositions, each being readily dispersible into the base compositions and each disposed in a separate dispensing container, the plurality of water dispersible cosmeticity booster compositions comprising active ingredients corresponding to hair target attributes that include exfoliating, volumizing, plumping, smoothing, softening, repairing/softening, and scenting, the plurality of water dispersible cosmeticity booster compositions comprising two or more of:
i) the booster composition corresponding to exfoliating that includes a powder, at least one clay, at least one filler, and starch; and
ii) the booster composition corresponding to volumizing that includes sodium bicarbonate, an organic acid, and starch; and
iii) the booster composition corresponding to plumping that includes at least one polymeric thickener, in some embodiments comprising a polysaccharide, in some embodiments comprising a polysaccharide gellifying thickener, together with aloe, one or a combination of polyols, and optionally an active comprising an organic acid; and
iv) the booster composition corresponding to smoothing that includes at least one gum, and at least one hydroxy group containing compound selected from polyols, longer chain monoalcohols (C₈ to C₃₀) which include fatty alcohols, and combinations thereof; and
v) the booster composition corresponding to repairing butter that includes an anhydrous blend of plant/vegetable oils, and optionally at least one active comprising a vitamin; and
vi) the booster composition corresponding to repairing/softening oil that includes an anhydrous blend comprising at least one plant oil, at least one polar emollient ester, and optionally at least one active comprising a vitamin; and
vii) the booster composition corresponding to scenting that includes an essentially or anhydrous blend of a fragrance with at least one polar emollient selected from isopropyl palmitate, isopropyl myristate, and combinations thereof.
(II) Preparing a hair care formulation, including:
d) Selecting a first hair care formulation recipe from the hair product formulary schedule;
e) Dispensing into a mixing vessel a number of units of the aqueous base composition as set forth in the recipe;
f) Dispensing into the mixing vessel a number of units of each of one or a combination of booster compositions as set forth in the recipe, alternately mixing after addition of each booster composition; and
g) Completing any additional processing step as set forth in the recipe;
(III) Delivery of hair care, including:
h) Applying the hair care formulation to one of the scalp, the hair ends, the hair roots, and combinations thereof.

15) The method for delivering hair care according to claim 14, wherein the steps are repeated at least two times to form at least a pair of hair care formulations selected from at least one cleansing and at least one conditioning product, or at least two cleansing products, or at least two conditioning products.

16) A system for formulating a hair care product, comprising:
a) a hair product formulary schedule comprising a plurality of hair care formulation recipes, each hair care formulation recipe including instructions for combining one of a plurality of aqueous base compositions, and one or a combination of water dispersible cosmeticity booster compositions;
b) a plurality of aqueous base compositions comprising at least one cleansing base and at least one conditioning base, each aqueous base composition disposed in a separate dispensing container, the dispensing containers being configured for delivery of the compositions by actuation of a pump or squeeze action; and
c) a plurality of water dispersible cosmeticity booster compositions, each being readily dispersible into the base compositions and each disposed in a separate dispensing container, the plurality of water dispersible cosmeticity booster compositions comprising active ingredients corresponding to hair target attributes, the booster compositions provided in one of a liquidous, gel, serum, or solid powderous form, the liquidous, gel and serum dispensing containers being configured for delivery of the compositions by actuation of a pump or squeeze action, and the powderous dispensing containers being configured as packets comprising a fixed unit dispensed amount.
